(19) United States
(12) Patent Application Publication
(10) Pub. No.: US 2023/0381286 A1
(43) Pub. Date: Nov. 30, 2023

(54) AFFINITY-BASED METHODS FOR USING TRANSFERRIN RECEPTOR-BINDING PROTEINS

(71) Applicant: Denali Therapeutics Inc., South San Francisco, CA (US)

(72) Inventors: Mark S. Dennis, South San Francisco, CA (US); Jennifer Getz, South San Francisco, CA (US); Mihalis Kariolis, South San Francisco, CA (US); Adam P. Silverman, South San Francisco, CA (US); Robert C. Wells, South San Francisco, CA (US); Joy Yu Zuchero, South San Francisco, CA (US)

(73) Assignee: Denali Therapeutics Inc., South San Francisco, CA (US)

(21) Appl. No.: 18/166,223

(22) Filed: Feb. 8, 2023

Related U.S. Application Data
(63) Continuation of application No. 16/782,984, filed on Feb. 5, 2020, now abandoned.
Application No. 16/782,984 is a continuation of application No. PCT/US2018/046337, filed on Aug. 10, 2018.
(60) Provisional application No. 62/543,658, filed on Aug. 10, 2017.
(60) Provisional application No. 62/583,314, filed on Nov. 8, 2017.

(30) Foreign Application Priority Data
Feb. 15, 2018 (WO) PCT/US2018/018371

(51) Int. Cl. A61K 38/40; A61K 47/64; C07K 16/28; C07K 14/79

(52) U.S. Cl. CPC A61K 38/40; A61K 47/644; C07K 16/2881; C07K 14/79; C07K 2317/52; C07K 2317/526; C07K 2317/94; A61K 38/00

(57) ABSTRACT

Provided herein are methods for transporting agents across the blood brain barrier. In some embodiments, the agents bind to therapeutic targets for the treatment of neurodegenerative diseases. As described herein, the agents are linked to proteins that bind to a transferrin receptor.

### CROSS-REFERENCE TO RELATED APPLICATIONS

The present application is a continuation of U.S. patent application Ser. No. 16/782,984, filed Feb. 5, 2020, which is a continuation of International Patent Application No. PCT/US2018/046337, filed Aug. 10, 2018, which claims priority to International Patent Application No. PCT/US2018/018371, filed on Feb. 15, 2018, U.S. Provisional Application No. 62/583,314, filed on Nov. 8, 2017, and U.S. Provisional Application No. 62/543,658, filed on Aug. 10, 2017, the disclosures of which are incorporated herein by reference in their entirety for all purposes.

### SEQUENCE LISTING

A Sequence Listing conforming to the rules of WIPO Standard ST.26 is hereby incorporated by reference. Said Sequence Listing has been filed as an electronic document via PatentCenter in ASCII format encoded as XML. The electronic document, created on Feb. 7, 2023, is entitled “102342-1371968-002710US_ST26.xml”, and is 7,497 bytes in size.

### BRIEF DESCRIPTION OF THE DRAWINGS

FIG. 1 shows pharmacokinetic (PK) analysis for CH3C polypeptides in wild-type mice. All polypeptide-Fab fusions had comparable clearance to wild-type Fc-Fab fusions (i.e., Ab122, an anti-RSV antibody, and Ab153, an anti-BACE1 antibody) except CH3C.3.2-5, which had faster clearance.

FIG. 2 shows brain pharmacokinetic/pharmacodynamic (PK/PD) data in mouse brain tissue. Chimeric huTfR heterozygous mice (n=4/group) were intravenously dosed with 42 mg/kg of either Ab153 or monovalent CH3C.35.N163 (labeled “CH3C.35.N163_mono”), and wild-type mice (n=3) were dosed intravenously with 50 mg/kg of control human IgG1 (labeled “huIgG1”). Bar graphs represent mean+/−SD.

FIGS. 3A and 3B depict huIgG1 concentrations in plasma (FIG. 3A) and brain lysates (FIG. 3B) of hTfR^{apical+/+} knock-in (KI) mice after a single 50 mg/kg systemic injection of anti-BACE1_Ab153, CH3C35.21:Ab153, CH3C35.20:Ab153, or CH3C35:Ab153 polypeptide fusion (mean±SEM, n=5 per group).

FIG. 3C depicts endogenous mouse Aβ concentration in brain lysate of hTfR^{apical+/+} KI mice after a single 50 mg/kg systemic injection of anti-BACE1_Ab153, CH3C35.21:Ab153, CH3C35.20:Ab153, or CH3C35:Ab153 polypeptide fusion (mean±SEM, n=5 per group).

FIG. 3D depicts Western blot quantification of brain TfR protein normalized to actin in brain lysate of hTfR^{apical+/+} KI mice after a single 50 mg/kg systemic injection of anti-BACE1_Ab153, CH3C35.21:Ab153, CH3C35.20:Ab153, or CH3C35:Ab153 polypeptide fusion (mean±SEM, n=5 per group).

FIGS. 4A and 4B depict huIgG1 concentrations in plasma (FIG. 4A) and brain lysates (FIG. 4B) of hTfR^{apical+/+} KI mice after a single 50 mg/kg systemic injection of anti-BACE1_Ab153, CH3C.35.23:Ab153, or CH3C.35.23.3:Ab153 polypeptide fusion (mean SEM, n=5 per group).

FIG. 4C depicts endogenous mouse Aβ concentration in brain lysate of hTfR^{apical+/+} KI mice after a single 50 mg/kg systemic injection of anti-BACE1_Ab153, CH3C.35.23:Ab153, or CH3C.35.23.3:Ab153 polypeptide fusion (mean±SEM, n=5 per group).

FIG. 4D depicts Western blot quantification of brain TfR protein normalized to actin in brain lysate of hTfR^{apical+/+} KI mice after a single 50 mg/kg systemic injection of anti-BACE1_Ab153, CH3C.35.23:Ab153, or CH3C.35.23.3:Ab153 polypeptide fusion (mean±SEM, n=4 per group).

FIG. 5 shows the relationship between engineered TfR-binding polypeptide hTfR affinity and brain exposure over time in hTfR^{apical+/+} KI mice. Dots represent cumulative brain exposure over time (AUC) of different ATV affinity variants following a single dose of 50 mg/kg in hTfR^{apical+/+} KI mice. Brain concentrations of polypeptide (as measured by huIgG1) were calculated at various days post-dose (ranges from 1-10 days). Data represents summary of three independent studies, n=4-5 mice per group for each study.

FIG. 6 shows the relationship between engineered TfR-binding polypeptide hTfR affinity and maximum brain concentration in hTfR^{apical+/+} KI mice. Dots represent maximum brain concentrations of different polypeptide affinity variants measured at 1 day post-dose after a single 50 mg/kg dose. Data represents summary of three independent studies, n=4-5 mice per group for each study.

FIG. 7 shows the relationship between between engineered TfR-binding polypeptide hTfR affinity and ratio of brain versus plasma concentration of polypeptide in hTfR^{apical+/+} KI mice. Dots represent ratio of maximum brain versus plasma concentration of different polypeptide affinity variants measured at 1 day post-dose after a single 50 mg/kg dose. Data represents summary of three independent studies, n=4-5 mice per group for each study.

### DETAILED DESCRIPTION OF THE INVENTION

### I. Introduction

The present invention relates to transporting therapeutic agents that are linked to TfR-binding polypeptides and proteins across the blood brain barrier (BBB) for the treatment of disease. The present invention is based, in part, on the discovery that the desired TfR binding affinity for transporting a therapeutic agent across the BBB depends on the target of the therapeutic agent, as well as the mechanism of action that drives efficacy in treating the disease. In particular, it has been discovered that using polypeptides and proteins that have relatively lower TfR affinities results in lower Cₘₐₓ but slower clearance, which results in prolonged exposure.

For some therapies, which include the use of inhibitory agents, including inhitibtory antibodies such as anti-BACE1 and anti-Tau agents (e.g., for the treatment of Alzheimer's disease) and anti-alpha-synuclein agents (e.g., for the treatment of Parkinson's disease), as well as others, achieving prolonged or sustained brain exposure of the therapeutic agent is desired over the dosing window in order to engage targets fully, including targets that have a short half-life and/or fast turnover (e.g., Tau, alpha-synuclein), and to sustain inhibition of BACE1 activity in order to reduce Abeta production (which also has a short half-life). For achieving prolonged or sustained brain exposure to a therapeutic agent, using polypeptides and proteins that have a TfR affinity range of 400-2,000 nM is particularly useful.

### II. Definitions

As used herein, the singular forms “a,” “an,” and “the” include plural referents unless the content clearly dictates otherwise. Thus, for example, reference to “a polypeptide” may include two or more such molecules, and the like.

As used herein, the terms “about” and “approximately,” when used to modify an amount specified in a numeric value or range, indicate that the numeric value as well as reasonable deviations from the value known to the skilled person in the art, for example 20%, +10%, or +5%, are within the intended meaning of the recited value.

A “transferrin receptor” or “TfR” as used in the context of this invention refers to transferrin receptor protein 1. The human transferrin receptor 1 polypeptide sequence is set forth in SEQ ID NO:6. Transferrin receptor protein 1 sequences from other species are also known (e.g., chimpanzee, accession number XP_003310238.1; rhesus monkey, NP_001244232.1; dog, NP_001003111.1; cattle, NP_001193506.1; mouse, NP_035768.1; rat, NP_073203.1; and chicken, NP_990587.1). The term “transferrin receptor” also encompasses allelic variants of exemplary reference sequences, e.g., human sequences, that are encoded by a gene at a transferrin receptor protein 1 chromosomal locus. Full length transferrin receptor protein includes a short N-terminal intracellular region, a transmembrane region, and a large extracellular domain. The extracellular domain is characterized by three domains: a protease-like domain, a helical domain, and an apical domain. The apical domain sequence of human transferrin receptor 1 is set forth in SEQ ID NO:4.

As used herein, the term “Fc polypeptide” refers to the C-terminal region of a naturally occurring immunoglobulin heavy chain polypeptide that is characterized by an Ig fold as a structural domain. An Fc polypeptide contains constant region sequences including at least the CH2 domain and/or the CH3 domain and may contain at least part of the hinge region. In general, an Fc polypeptide does not contain a variable region.

A “modified Fc polypeptide” refers to an Fc polypeptide that has at least one mutation, e.g., a substitution, deletion or insertion, as compared to a wild-type immunoglobulin heavy chain Fc polypeptide sequence, but retains the overall Ig fold or structure of the native Fc polypeptide.

The terms “CH3 domain” and “CH2 domain” as used herein refer to immunoglobulin constant region domain polypeptides. In the context of IgG antibodies, a CH3 domain polypeptide refers to the segment of amino acids from about position 341 to about position 447 as numbered according to the EU numbering scheme, and a CH2 domain polypeptide refers to the segment of amino acids from about position 231 to about position 340 as numbered according to the EU numbering scheme. CH2 and CH3 domain polypeptides may also be numbered by the IMGT (ImMunoGeneTics) numbering scheme in which the CH2 domain numbering is 1-110 and the CH3 domain numbering is 1-107, according to the IMGT Scientific chart numbering (IMGT website). CH2 and CH3 domains are part of the Fc region of an immunoglobulin. In the context of IgG antibodies, an Fc region refers to the segment of amino acids from about position 231 to about position 447 as numbered according to the EU numbering scheme. As used herein, the term “Fc region” may also include at least a part of a hinge region of an antibody. An illustrative hinge region sequence is set forth in SEQ ID NO:5.

The term “variable region” refers to a domain in an antibody heavy chain or light chain derived from a germline Variable (V) gene, Diversity (D) gene, or Joining (J) gene (and not derived from a Constant (C and CS) gene segment), and that gives an antibody its specificity for binding to an antigen. Typically, an antibody variable region comprises four conserved “framework” regions interspersed with three hypervariable “complementarity determining regions.”

The terms “wild-type,” “native,” and “naturally occurring” with respect to a CH3 or CH2 domain are used herein to refer to a domain that has a sequence that occurs in nature.

In the context of this invention, the term “mutant” with respect to a mutant polypeptide or mutant polynucleotide is used interchangeably with “variant.” A variant with respect to a given wild-type (e.g., CH3 or CH2 domain) reference sequence can include naturally occurring allelic variants. A “non-naturally” occurring (e.g., CH3 or CH2) domain refers to a variant or mutant domain that is not present in a cell in nature and that is produced by genetic modification, e.g., using genetic engineering technology or mutagenesis techniques, of a native domain (e.g., CH3 domain or CH2 domain) polynucleotide or polypeptide. A “variant” includes any domain comprising at least one amino acid mutation with respect to wild-type. Mutations may include substitutions, insertions, and deletions.

The term “binding affinity” as used herein refers to the strength of the non-covalent interaction between two molecules, e.g., a single binding site on a polypeptide and a target, e.g., TfR, to which it binds. Thus, for example, the term may refer to 1:1 interactions between a polypeptide and its target, unless otherwise indicated or clear from context. Binding affinity may be quantified by measuring an equilibrium dissociation constant (K_{D}), which refers to the dissociation rate constant (k_{d}, time⁻¹) divided by the association rate constant (kₐ, time⁻¹ M⁻¹). K_{D} can be determined by measurement of the kinetics of complex formation and dissociation, e.g., using Surface Plasmon Resonance (SPR) methods, e.g., a Biacore™ system (for example, using the method described in Example 3 below); kinetic exclusion assays such as KinExA®; and BioLayer interferometry (e.g., using the ForteBio® Octet® platform). As used herein, “binding affinity” includes not only formal binding affinities, such as those reflecting 1:1 interactions between a polypeptide and its target, but also apparent affinities for which K_{D}'s are calculated that may reflect avid binding.

As used herein, the term “specifically binds” or “selectively binds” to a target, e.g., TfR, when referring to an engineered TfR-binding polypeptide, TfR-binding peptide, or TfR-binding antibody as described herein, refers to a binding reaction whereby the engineered TfR-binding polypeptide, TfR-binding peptide, or TfR-binding antibody binds to the target with greater affinity, greater avidity, and/or greater duration than it binds to a structurally different target. In typical embodiments, the engineered TfR-binding polypeptide, TfR-binding peptide, or TfR-binding antibody has at least 5-fold, 6-fold, 7-fold, 8-fold, 9-fold, 10-fold, 20-fold, 25-fold, 50-fold, 100-fold, 1,000-fold, 10,000-fold, or greater affinity for a specific target, e.g., TfR, compared to an unrelated target when assayed under the same affinity assay conditions. The term “specific binding,” “specifically binds to,” or “is specific for” a particular target (e.g., TfR), as used herein, can be exhibited, for example, by a molecule having an equilibrium dissociation constant K_{D} for the target to which it binds of, e.g., 10⁻⁴ M or smaller, e.g., 10⁻⁵ M, 10⁻⁶ M, 10⁻⁷ M, 10⁻¹ M, 10⁻⁹ M, 10⁻¹⁰ M, 10⁻¹¹ M, or 10⁻¹² M. In some embodiments, an engineered TfR-binding polypeptide, TfR-binding peptide, or TfR-binding antibody specifically binds to an epitope on TfR that is conserved among species, (e.g., structurally conserved among species), e.g., conserved between non-human primate and human species (e.g., structurally conserved between non-human primate and human species). In some embodiments, an engineered TfR-binding polypeptide, TfR-binding peptide, or TfR-binding antibody may bind exclusively to a human TfR.

The term “amino acid” refers to naturally occurring and synthetic amino acids, as well as amino acid analogs and amino acid mimetics that function in a manner similar to the naturally occurring amino acids.

Naturally occurring amino acids are those encoded by the genetic code, as well as those amino acids that are later modified, e.g., hydroxyproline, γ-carboxyglutamate and O-phosphoserine. “Amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that function in a manner similar to a naturally occurring amino acid.

Naturally occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of naturally occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Amino acids may be referred to herein by either their commonly known three letter symbols or by the one-letter symbols recommended by the IUPAC-IUB Biochemical Nomenclature Commission.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids.

The term “protein” as used herein refers to either a polypeptide or a dimer (i.e., two) or multimer (i.e., three or more) of single chain polypeptides. The single chain polypeptides of a protein may be joined by a covalent bond, e.g., a disulfide bond, or non-covalent interactions.

The term “conservative substitution,” “conservative mutation,” or “conservatively modified variant” refers to an alteration that results in the substitution of an amino acid with another amino acid that can be categorized as having a similar feature. Examples of categories of conservative amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R), and His (Histidine or H); an “aromatic group” including Phe (Phenylalanine or F), Tyr (Tyrosine or Y), Trp (Tryptophan or W), and (Histidine or H); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T), and Cys (Cysteine or C). Within each group, subgroups can also be identified. For example, the group of charged or polar amino acids can be sub-divided into sub-groups including: a “positively-charged sub-group” comprising Lys, Arg and His; a “negatively-charged sub-group” comprising Glu and Asp; and a “polar sub-group” comprising Asn and Gln. In another example, the aromatic or cyclic group can be sub-divided into sub-groups including: a “nitrogen ring sub-group” comprising Pro, His and Trp; and a “phenyl sub-group” comprising Phe and Tyr. In another further example, the aliphatic group can be sub-divided into sub-groups, e.g., an “aliphatic non-polar sub-group” comprising Val, Leu, Gly, and Ala; and an “aliphatic slightly-polar sub-group” comprising Met, Ser, Thr, and Cys. Examples of categories of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH₂ can be maintained. In some embodiments, hydrophobic amino acids are substituted for naturally occurring hydrophobic amino acids, e.g., in the active site, to preserve hydrophobicity.

The terms “identical” or percent “identity,” in the context of two or more polypeptide sequences, refer to two or more sequences or subsequences that are the same or have a specified percentage of amino acid residues, e.g., at least 60%, at least 65%, at least 70%, at least 75%, at least 80%, at least 85%, at least 90%, or at least 95% or greater, that are identical over a specified region when compared and aligned for maximum correspondence over a comparison window or designated region, as measured using a sequence comparison algorithm or by manual alignment and visual inspection.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a modified Fc polypeptide “corresponds to” an amino acid in SEQ ID NO:1, when the residue aligns with the amino acid in SEQ ID NO:1 when optimally aligned to SEQ ID NO: 1. The polypeptide that is aligned to the reference sequence need not be the same length as the reference sequence.

The term “subject,” “individual,” and “patient,” as used interchangeably herein, refer to a mammal, including but not limited to humans, non-human primates, rodents (e.g., rats, mice, and guinea pigs), rabbits, cows, pigs, horses, and other mammalian species. In one embodiment, the patient is a human.

The terms “treatment,” “treating,” and the like are used herein to generally mean obtaining a desired pharmacologic and/or physiologic effect. “Treating” or “treatment” may refer to any indicia of success in the treatment or amelioration of an injury, disease, or condition, including any objective or subjective parameter such as abatement, remission, improvement in patient survival, increase in survival time or rate, diminishing of symptoms or making the injury, disease, or condition more tolerable to the patient, slowing in the rate of degeneration or decline, or improving a patient's physical or mental well-being. The treatment or amelioration of symptoms can be based on objective or subjective parameters. The effect of treatment can be compared to an individual or pool of individuals not receiving the treatment, or to the same patient prior to treatment or at a different time during treatment.

The term “pharmaceutically acceptable excipient” refers to a non-active pharmaceutical ingredient that is biologically or pharmacologically compatible for use in humans or animals, such as but not limited to a buffer, carrier, or preservative.

As used herein, a “therapeutic amount,” “therapeutically effective amount,” or “therapeutically effective concentration” of an agent is an amount or concentration of the agent that treats signs or symptoms of a disease in the subject.

The term “administer” refers to a method of delivering agents, compounds, or compositions to the desired site of biological action. These methods include, but are not limited to, topical delivery, parenteral delivery, intravenous delivery, intradermal delivery, intramuscular delivery, intrathecal delivery, colonic delivery, rectal delivery, or intraperitoneal delivery. In one embodiment, the compositions described herein are administered intravenously.

### III. Therapeutic Methods

A. Methods for Treating Neurodegenerative Diseases

In one aspect, the present invention provides a method for transporting an agent (e.g., therapeutic agent) that binds (e.g., specifically binds) to a therapeutic target (e.g., a therapeutic target implicated in a neurodegenerative disease) across the blood-brain barrier (BBB) of a mammal. In some embodiments, the method comprises exposing the BBB to a polypeptide or protein that binds (e.g., specifically binds) to a transferrin receptor (TfR) with an affinity of from about 400 nM to about 2 μM. In some embodiments, the polypeptide or protein is linked to the agent and transports the linked agent across the BBB. In some embodiments, brain exposure to the agent is prolonged (e.g., as compared to a reference).

In another aspect, the present invention provides a method for treating a neurodegenerative disease. In some embodiments, the method comprises administering to a mammal a polypeptide or protein that binds (e.g., specifically binds) to a TfR with an affinity of from about 400 nM to about 2 μM. In some embodiments, the polypeptide or protein is linked to an agent (e.g., therapeutic agent) that binds (e.g., specifically binds) to a therapeutic target implicated in the neurodegenerative disease, thereby prolonging exposure of the brain of the mammal to the agent. Non-limiting examples of suitable neurodegenerative diseases include Alzheimer's disease (AD), Parkinson's disease, amyotrophic lateral sclerosis (ALS), and a combination thereof.

In some embodiments, the polypeptide or protein binds (e.g., specifically binds) to a TfR with an affinity of about 400 nM, 500 nM, 600 nM, 700 nM, 800 nM, 900 nM, 1 μM, 1.1 μM, 1.2 μM, 1.3 μM, 1.4 μM, 1.5 μM, 1.6 μM, 1.7 μM, 1.8 μM, 1.9 μM, or 2 μM. In some embodiments, the polypeptide or protein binds to a TfR with an affinity of from about 420 nM to about 1.5 μM or 600 nM to 1.5 μM. In some embodiments, the polypeptide or protein binds to a TfR with an affinity of about 420 nM. In some embodiments, the polypeptide or protein binds to a TfR with an affinity of about 620 nM. In some embodiments, the polypeptide or protein binds to a TfR with an affinity of about 750 nM. In some embodiments, the polypeptide or protein binds to a TfR with an affinity of about 820 nM. In some embodiments, the polypeptide or protein binds to a TfR with an affinity of about 1,100 nM. In some embodiments, the polypeptide or protein binds to a TfR with an affinity of about 1,440 nM.

In some embodiments, the polypeptide or protein (e.g., linked to the agent) prolongs brain exposure to the agent at a therapeutically effective concentration (e.g., a concentration that is sufficient to treat one or more signs or symptoms of a neurodegenerative disease) in the mammal as compared to the agent linked to a reference polypeptide or protein that binds (e.g., specifically binds) to the TfR with a stronger affinity.

In some embodiments, brain exposure (e.g., to the agent) is prolonged by at least about 1.1-fold, 1.2-fold, 1.3-fold, 1.4-fold, 1.5-fold, 1.75-fold, 2-fold, 2.5-fold, 3-fold, 5-fold, or more, as compared to a reference.

In some embodiments, brain exposure is quantified by plotting brain exposure (e.g., concentration of the agent in the brain) as a function of time and calculating the area under the curve (AUC). Increased AUC can represent increased or prolonged brain exposure. In some embodiments, duration of brain exposure to the agent at a therapeutically effective concentration is increased.

In some embodiments, the reference polypeptide or protein binds (e.g., specifically binds) to the TfR with an affinity that is about, or is stronger than about, 400 nM, 350 nM, 300 nM, 250 nM, 200 nM, 150 nM, 100 nM, or 50 nM. In some embodiments, the reference polypeptide or protein binds to the TfR with an affinity that is about, or is stronger than about, 50 nM.

As non-limiting examples, the therapeutic target may be a target such as a beta-secretase 1 (BACE1) protein, a Tau protein, a triggering receptor expressed on myeloid cells 2 (TREM2) protein, or an alpha-synuclein protein. In some embodiments, the therapeutic target is BACE1 and the agent (e.g., therapeutic agent) decreases the amount of amyloid beta-protein (Abeta) that is present in the brain of the mammal for a longer duration when linked to the protein as compared to when the agent is linked to the reference protein.

In some embodiments, the mammal is a primate (e.g., a human). In some embodiments, the human is a patient in need of treatment for a neurological disease (e.g., a neurodegenerative disease). In some embodiments, the patient has one or more signs or symptoms of a neurological disease.

In some embodiments, the polypeptide or protein binds (e.g., specifically binds) to a primate TfR. In some embodiments, the primate TfR is a human TfR. In some embodiments, the polypeptide or protein binds to a TfR apical domain.

In some embodiments, the agent (e.g., therapeutic agent) is linked to an engineered TfR-binding polypeptide. In some embodiments, the engineered TfR-binding polypeptide comprises CH3 or CH2 domains that have modifications that allow the polypeptide to specifically bind to a transferrin receptor. Non-limiting examples of suitable engineered TfR-biding polypeptides are described in Section IV below. In some embodiments, the agent is linked to an engineered TfR-binding polypeptide that is described in Table 1 or Table 2. In some embodiments, the agent is linked to an engineered TfR-binding polypeptide selected from the group consisting of CH3C.35.23, CH3C.35.23.1.1, CH3C.35.23.3, and CH3C.35.23.4.

In some embodiments, the agent (e.g., therapeutic agent) is linked to a TfR-binding peptide. In some embodiments, the TfR-binding peptide is a short peptide, being about 5, 6, 7, 8, 9, 10, 11, 12, 13, 14, 15, 16, 17, 18, 19, or 20 amino acids in length. Methods for generating, screening, and identifying suitable peptides (i.e., that bind to a TfR with an affinity within the desired range) are known in the art. For example, a phage display strategy in which alternating rounds of negative and positive selection are employed can be used to identify suitable peptides. This strategy is described, e.g., in Lee et al., Eur. J Biochem. (2001) 268:2004-2012, which is hereby incorporated in its entirety for all purposes.

In some embodiments, the agent (e.g., therapeutic agent) is linked to a TfR-binding antibody. A non-limiting example of a suitable TfR-binding antibody is the H67 antibody disclosed in Chinese Patent Application Publication No. CN101245107A, which has an affinity of about 480 nM.

In some embodiments, the protein comprises an antibody variable region that specifically binds to TfR. In some instances, the protein comprises an antibody fragment. In some instances, the protein comprises a Fab or an scFv.

In some embodiments, the agent (e.g., therapeutic agent) comprises an antibody variable region. In some embodiments, the agent comprises an antibody fragment. In some embodiments, the agent comprises a Fab or an scFv.

In some embodiments, the agent (e.g., therapeutic agent) comprises a Fab and the polypeptide is in an Fc format (which may contain a hinge or partial hinge region), thus generating a transferrin receptor-binding Fc-Fab fusion. In some embodiments, an Fc-Fab fusion (e.g., comprising a modified CH2 or CH3 domain polypeptide) is a subunit of a dimer. In some embodiments, the dimer is a heterodimer. In some embodiments, the dimer is a homodimer. In some embodiments, the dimer comprises a single polypeptide that binds to the transferrin receptor, i.e., is monovalent for transferrin receptor binding. In some embodiments, the dimer comprises a second polypeptide that binds to the transferrin receptor. The second polypeptide may comprise the same modified CH3 domain polypeptide (or modified CH2 domain polypeptide) present in the Fc-Fab fusion to provide a bivalent binding homodimer, or a second modified CH3 domain polypeptide (or modified CH2 domain polypeptide) may provide a second transferrin receptor binding site. In some embodiments, the dimer comprises a first subunit comprising a modified CH3 domain polypeptide or modified CH2 domain polypeptide and a second subunit comprising CH2 and CH3 domains where neither binds a transferrin receptor.

In some embodiments, an agent (e.g., a Fab fragment) is linked to the polypeptide or protein and binds to a Tau protein (e.g., a human Tau protein) or a fragment thereof. In some embodiments, the agent may bind to a phosphorylated Tau protein, an unphosphorylated Tau protein, a splice isoform of Tau protein, an N-terminal truncated Tau protein, a C-terminal truncated Tau protein, and/or a fragment thereof.

In some embodiments, an agent (e.g., a Fab fragment) is linked to the polypeptide or protein and binds to a beta-secretase 1 (BACE1) protein (e.g., a human BACE1 protein) or a fragment thereof. In some embodiments, the agent may bind to one or more splice isoforms of BACE1 protein or a fragment thereof.

In some embodiments, an agent (e.g., a Fab fragment) is linked to the polypeptide or protein and binds to a triggering receptor expressed on myeloid cells 2 (TREM2) protein (e.g., a human TREM2 protein) or a fragment thereof.

In some embodiments, an agent (e.g., a Fab fragment) is linked to the polypeptide or protein and binds to an alpha-synuclein protein (e.g., a human alpha-synuclein protein) or a fragment thereof. In some embodiments, the agent may bind to a monomeric alpha-synuclein, oligomeric alpha-synuclein, alpha-synuclein fibrils, soluble alpha-synuclein, and/or a fragment thereof.

B. Additional Embodiments and Linkers

A polypeptide (e.g., a modified CH3 or CH2 domain polypeptide as described further below) may be joined to another domain of an Fc region. In some embodiments, a modified CH3 domain polypeptide is joined to a CH2 domain, which may be a naturally occurring CH2 domain or a variant CH2 domain, typically at the C-terminal end of the CH2 domain. In some embodiments, a modified CH2 domain polypeptide is joined to a CH3 domain, which may be a naturally occurring CH3 domain or a CH3 variant domain, typically at the N-terminal end of the CH3 domain. In some embodiments, the polypeptide comprising a modified CH2 domain joined to a CH3 domain, or the polypeptide comprising the modified CH3 domain joined to a CH2 domain, further comprises a partial or full hinge region of an antibody, thus resulting in a format in which the modified CH3 domain polypeptide or modified CH2 domain polypeptide is part of an Fc region having a partial or full hinge region. The hinge region can be from any immunoglobulin subclass or isotype. An illustrative immunoglobulin hinge is an IgG hinge region, such as an IgG1 hinge region, e.g., human IgG1 hinge amino acid sequence EPKSCDKTHTCPPCP (SEQ ID NO:5).

In still other embodiments, the engineered TfR-binding polypeptide, TfR-binding peptide, or TfR-binding antibody may be fused to a peptide or protein useful in protein purification, e.g., polyhistidine, epitope tags, e.g., FLAG, c-Myc, hemagglutinin tags and the like, glutathione S transferase (GST), thioredoxin, protein A, protein G, or maltose binding protein (MBP). In some cases, the peptide or protein to which the engineered TfR-binding polypeptide, TfR-binding peptide, or TfR-binding antibody is fused may comprise a protease cleavage site, such as a cleavage site for Factor Xa or Thrombin.

In methods of the present invention, an agent (e.g., therapeutic agent) is linked to a polypeptide or protein (e.g., an engineered TfR-binding polypeptide, a TfR-binding peptide, or a TfR-binding antibody). The linker may be any linker suitable for joining an agent to the polypeptide or protein. In some embodiments, the linkage is enzymatically cleavable. In certain embodiments, the linkage is cleavable by an enzyme present in the central nervous system.

In some embodiments, the linker is a peptide linker. The peptide linker may be configured such that it allows for the rotation of the agent (e.g., therapeutic agent) and the polypeptide or protein relative to each other; and/or is resistant to digestion by proteases. In some embodiments, the linker may be a flexible linker, e.g., containing amino acids such as Gly, Asn, Ser, Thr, Ala, and the like. Such linkers are designed using known parameters. For example, the linker may have repeats, such as Gly-Ser repeats.

In various embodiments, linking of the agent (e.g., therapeutic agent) to the polypeptide or protein (e.g., engineered TfR-binding polypeptide, TfR-binding peptide, or TfR-binding antibody) can be achieved using well-known chemical cross-linking reagents and protocols. For example, there are a large number of chemical cross-linking agents that are known to those skilled in the art and useful for cross-linking the polypeptide or protein with an agent of interest. For example, the cross-linking agents are heterobifunctional cross-linkers, which can be used to link molecules in a stepwise manner. Heterobifunctional cross-linkers provide the ability to design more specific coupling methods for conjugating proteins, thereby reducing the occurrences of unwanted side reactions such as homo-protein polymers.

The agent (e.g., therapeutic agent) may be linked to the N-terminal or C-terminal region of the polypeptide or protein, or attached to any region of the polypeptide or protein (e.g., engineered TfR-binding polypeptide, TfR-binding peptide, or TfR-binding antibody), so long as the agent does not interfere with binding of the polypeptide or protein to a transferrin receptor.

C. Measuring Binding Affinity, Brain Concentration, and Brain Exposure

In some embodiments, the affinity of a TfR-binding polypeptide may be measured in a monovalent format. In other embodiments, affinity may be measured in a bivalent format, e.g., as a dimer comprising a polypeptide-Fab fusion protein.

Methods for analyzing binding affinity, binding kinetics, and cross-reactivity are known in the art. These methods include, but are not limited to, solid-phase binding assays (e.g., ELISA assay), immunoprecipitation, surface plasmon resonance (e.g., Biacore™ (GE Healthcare, Piscataway, NJ)), kinetic exclusion assays (e.g., KinExA®), flow cytometry, fluorescence-activated cell sorting (FACS), BioLayer interferometry (e.g., Octet® (ForteBio, Inc., Menlo Park, CA)), and Western blot analysis. In some embodiments, ELISA is used to determine binding affinity and/or cross-reactivity. In some embodiments, surface plasmon resonance (SPR) is used to determine binding affinity, binding kinetics, and/or cross-reactivity. In some embodiments, kinetic exclusion assays are used to determine binding affinity, binding kinetics, and/or cross-reactivity. In some embodiments, BioLayer interferometry assays are used to determine binding affinity, binding kinetics, and/or cross-reactivity.

A non-limiting example of a method for determining binding affinity (e.g., for TfR) is described in Example 3 below, in which a Bicaore™ instrument was used to determine affinity by surface plasmon resonance. In this method, an engineered TfR-binding polypeptide, a TfR-binding peptide, or a TfR-binding antibody of interest is captured on a sensor chip and serial dilutions of TfR are injected onto the sensor chip at a specified flow rate (e.g., 30 μL/min) and temperature (e.g., room temperature). Samples are analyzed using specified association and dissociation times (e.g., 45 and 180 seconds, respectively), followed by sensor chip regeneration. Binding responses are corrected by subtracting the measured response from a control (e.g., using an irrelevant IgG at similar density) and then steady-state affinities can be determined by using software to fit the equilibrium response against concentration.

The concentration of an agent (e.g., linked to an engineered TfR-binding polypeptide, a TfR-binding peptide, or a TfR-binding antibody) in the brain and/or plasma can be measured, for example, using a human transferrin receptor (hTfR) knock-in mouse model. Such a model can be used, for example, to measure and/or compare maximum brain concentration (Cₘₐₓ) and/or brain exposure, e.g., to determine whether Cₘₐₓ is increased and/or brain exposure is prolonged. The creation of a human apical TfR (hTfR^{apical+/+}) mouse knock-in model is described below in Example 2. To create a suitable model, a CRISPR/Cas9 system can be used to generate a mouse that expresses a human Tfrc apical domain within a murine Tfrc gene (e.g., in which in vivo expression is under the control of an endogenous promoter). In particular, Cas9, single guide RNAs and donor DNA (e.g., a human apical domain coding sequence that has been codon optimized for expression in mouse) can be introduced into mouse embryos (e.g., by pronuclear injection). The embryos can then be transferred to pseudo pregnant females. A founder male from the progeny of the female that received the embryos can be bred to wild-type females to generate F1 heterozygous mice. Homozygous mice can then be subsequently generated from breeding of F1 generation heterozygous mice.

For evaluation of brain and/or plasma concentration or exposure of the agent (e.g., linked to an engineered TfR-binding polypeptide, a TfR-binding peptide, or a TfR-binding antibody), the linked agent can be administered to the mouse model (e.g., hTfR^{apical+/+}) Plasma samples can be obtained from the mouse after a suitable period of time, followed by perfusion of the vascular system with a suitable solution. Following perfusion, brains (or portions thereof) can be extracted and homogenized and lysed, Concentrations of the agent in the plasma and/or brain lysate can then be determined using standard methods that will be known to one of ordinary skill in the art. By administering a range of doses to the knock-in mouse model, a standard curve can be generated. By administering to the knock-in mouse model an agent linked to different engineered TfR-binding polypeptides, TfR-binding peptides, or TfR-binding antibodies (e.g., having different TfR affinities), or an agent linked to a reference polypeptide or protein (e.g., that has a stronger affinity for TfR than the polypeptide or protein of interest), comparisons can be made regarding the effects of the engineered TfR-binding polypeptides, TfR-binding peptides, or TfR-binding antibodies on brain exposure to the agent and/or Cₘₐₓ values of the agent in the brain.

D. Pharmaceutical Compositions

Guidance for preparing formulations for use in the present invention can be found in any number of handbooks for pharmaceutical preparation and formulation that are known to those of skill in the art.

In some embodiments, the polypeptide or protein linked to the agent (e.g., therapeutic agent) is administered as part of a pharmaceutically acceptable carrier or excipient. A pharmaceutically acceptable carrier includes any solvent, dispersion medium, or coating that is physiologically compatible and that preferably does not interfere with or otherwise inhibit the activity of the active agent. Various pharmaceutically acceptable excipients are well-known.

In some embodiments, the carrier is suitable for intravenous, intrathecal, intracerebroventricular, intramuscular, oral, intraperitoneal, transdermal, topical, or subcutaneous administration. Pharmaceutically acceptable carriers can contain one or more physiologically acceptable compounds that act, for example, to stabilize the composition or to increase or decrease the absorption of the polypeptide. Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers. Other pharmaceutically acceptable carriers and their formulations are also available in the art.

The pharmaceutical compositions described herein can be manufactured in a manner that is known to those of skill in the art, e.g., by means of conventional mixing, dissolving, granulating, dragee-making, emulsifying, encapsulating, entrapping, or lyophilizing processes. The following methods and excipients are merely exemplary and are in no way limiting.

For oral administration, a polypeptide or protein linked to an agent (e.g., therapeutic agent) can be formulated by combining it with pharmaceutically acceptable carriers that are well-known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the polypeptides with a solid excipient, optionally grinding a resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone. If desired, disintegrating agents can be added, such as a cross-linked polyvinyl pyrrolidone, agar, or alginic acid or a salt thereof such as sodium alginate.

A polypeptide or protein linked to an agent (e.g., therapeutic agent) can be formulated for parenteral administration by injection, e.g., by bolus injection or continuous infusion. For injection, the polypeptides can be formulated into preparations by dissolving, suspending, or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers, and preservatives. In some embodiments, polypeptides can be formulated in aqueous solutions, preferably in physiologically compatible buffers such as Hanks's solution, Ringer's solution, or physiological saline buffer. Formulations for injection can be presented in unit dosage form, e.g., in ampules or in multi-dose containers, with an added preservative. The compositions can take such forms as suspensions, solutions, or emulsions in oily or aqueous vehicles, and can contain formulatory agents such as suspending, stabilizing, and/or dispersing agents.

Typically, a pharmaceutical composition for use in in vivo administration is sterile. Sterilization can be accomplished according to methods known in the art, e.g., heat sterilization, steam sterilization, sterile filtration, or irradiation.

### IV. Engineered Transferrin Receptor-Binding Polypeptides

This section describes non-limiting examples of engineered polypeptides that bind to a transferrin receptor and are capable of being transported across the blood-brain barrier (BBB).

In some embodiments, the engineered polypeptides comprise CH3 or CH2 domains that have modifications that allow the polypeptides to specifically bind to a transferrin receptor. The modifications are introduced into specified sets of amino acids that are present at the surface of the CH3 or CH2 domain. In some embodiments, polypeptides comprising modified CH3 or CH2 domains specifically bind to an epitope in the apical domain of the transferrin receptor.

One of skill understands that CH2 and CH3 domains of other immunoglobulin isotypes, e.g., IgM, IgA, IgE, IgD, etc. may be similarly modified by identifying the amino acids in those domains that correspond to sets (i)-(vi) described herein. Modifications may also be made to corresponding domains from immunoglobulins from other species, e.g., non-human primates, monkey, mouse, rat, rabbit, dog, pig, chicken, and the like.

### CH3 Transferrin Receptor-Binding Polypeptides

In some embodiments, the domain that is modified is a human Ig CH3 domain, such as an IgG CH3 domain. The CH3 domain can be of any IgG subtype, i.e., from IgG1, IgG2, IgG3, or IgG4. In the context of IgG antibodies, a CH3 domain refers to the segment of amino acids from about position 341 to about position 447 as numbered according to the EU numbering scheme. The positions in the CH3 domain for purposes of identifying the corresponding set of amino acid positions for transferrin receptor binding are determined with reference to SEQ ID NO:3 or determined with reference to amino acids 114-220 of SEQ ID NO:1 unless otherwise specified. Substitutions are also determined with reference to SEQ ID NO:1, i.e., an amino acid is considered to be a substitution relative to the amino acid at the corresponding position in SEQ ID NO:1. SEQ ID NO:1 includes a partial hinge region sequence, PCP, as amino acids 1-3. The numbering of the positions in the CH3 domain with reference to SEQ ID NO:1 includes the first three amino acids.

As indicated above, sets of residues of a CH3 domain that can be modified are numbered herein with reference to SEQ ID NO:1. Any CH3 domain, e.g., an IgG1, IgG2, IgG3, or IgG4 CH3 domain, may have modifications, e.g., amino acid substitutions, in one or more sets of residues that correspond to residues at the noted positions in SEQ ID NO:1. The positions of each of the IgG2, IgG3, and IgG4 sequences that correspond to any given position of SEQ ID NO:1 can be readily determined.

In one embodiment, a modified CH3 domain polypeptide that specifically binds transferrin receptor binds to the apical domain of the transferrin receptor at an epitope that comprises position 208 of the full length human transferrin receptor sequence (SEQ ID NO:6), which corresponds to position 11 of the human transferrin receptor apical domain sequence set forth in SEQ ID NO:4. SEQ ID NO:4 corresponds to amino acids 198-378 of the human transferrin receptor-1 uniprotein sequence P02786 (SEQ ID NO:6). In some embodiments, the modified CH3 domain polypeptide binds to the apical domain of the transferrin receptor at an epitope that comprises positions 158, 188, 199, 207, 208, 209, 210, 211, 212, 213, 214, 215, and/or 294 of the full length human transferrin receptor sequence (SEQ ID NO:6). The modified CH3 domain polypeptide may bind to the transferrin receptor without blocking or otherwise inhibiting binding of transferrin to the receptor. In some embodiments, binding of transferrin to TfR is not substantially inhibited. In some embodiments, binding of transferrin to TfR is inhibited by less than about 50% (e.g., less than about 45%, 40%, 35%, 30%, 25%, 20%, 15%, 10%, or 5%). In some embodiments, binding of transferrin to TfR is inhibited by less than about 20% (e.g., less than about 19%, 18%, 17%, 16%, 15%, 14%, 13%, 12%, 11%, 10%, 9%, 8%, 7%, 6%, 5%, 4%, 3%, 2%, or 1%). Illustrative CH3 domain polypeptides that exhibit this binding specificity include polypeptides having amino acid substitutions at positions 153, 157, 159, 160, 161, 162, 163, 186, 188, 189, and 194 as determined with reference to amino acids 114-220 of SEQ ID NO:1.

CH3 Transferrin Receptor Binding Set (i): 153, 157, 159, 160, 161, 162, 163, 186, 188, 189, and 194

In some embodiments, a modified CH3 domain polypeptide comprises 1, 2, 3, 4, 5, 6, 7, 8, 9, 10, or 11 substitutions in a set of amino acid positions comprising 153, 157, 159, 160, 161, 162, 163, 186, 188, 189, and 194 (set i). Illustrative substitutions that may be introduced at these positions are shown in Tables 1 and 2.

In some embodiments, a modified CH3 domain polypeptide that specifically binds a transferrin receptor comprises at least one position having a substitution, relative to SEQ ID NO:1, as follows: Glu, Leu, Ser, Val, Trp, Tyr, or Gln at position 153; Leu, Tyr, Phe, Trp, Met, Pro, or Val at position 157; Leu, Thr, His, Pro, Asn, Val, or Phe at position 159; Val, Pro, Ile, or an acidic amino acid at position 160; Trp at position 161; an aliphatic amino acid, Gly, Ser, Thr, or Asn at position 162; Gly, His, Gln, Leu, Lys, Val, Phe, Ser, Ala, Asp, Glu, Asn, Arg, or Thr at position 163; an acidic amino acid, Ala, Ser, Leu, Thr, Pro, Ile, or His at position 186; Glu, Ser, Asp, Gly, Thr, Pro, Gln, or Arg at position 188; Thr, Arg, Asn, or an acidic amino acid at position 189; and/or an aromatic amino acid, His, or Lys at position 194. In some embodiments, a modified CH3 domain polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set. Thus, for example, Ile may be present at position 157, 159, and/or position 186. In some embodiments, the acidic amino acid at position one, two, or each of positions 160, 186, and 189 is Glu. In other embodiments, the acidic amino acid at one, two or each of positions 160, 186, and 189 is Asp.

In some embodiments, the modified CH3 domain polypeptide further comprises one or two substitutions at positions comprising 164 and 165. In some embodiments, Ser, Thr, Gln, or Phe may be present at position 164. In some embodiments, Gln, Phe, or His may be present at position 165.

In additional embodiments, the modified CH3 domain further comprises one, two, or three positions selected from the following: position 187 is Lys, Arg, Gly, or Pro; position 197 is Ser, Thr, Glu, or Lys; and position 199 is Ser, Trp, or Gly.

CH3 Transferrin Receptor Binding Set (ii): 118, 119, 120, 122, 210, 211, 212, and 213

In some embodiments, a modified CH3 domain polypeptide comprises at least three or at least four, and typically five, six, seven, or eight substitutions in a set of amino acid positions comprising 118, 119, 120, 122, 210, 211, 212, and 213 (set ii). In some embodiments, the modified CH3 domain polypeptide comprises Gly at position 210; Phe at position 211; and/or Asp at position 213. In some embodiments, Glu is present at position 213. In certain embodiments, a modified CH3 domain polypeptide comprises at least one substitution at a position as follows: Phe or Ile at position 118; Asp, Glu, Gly, Ala, or Lys at position 119; Tyr, Met, Leu, Ile, or Asp at position 120; Thr or Ala at position 122; Gly at position 210; Phe at position 211; His Tyr, Ser, or Phe at position 212; or Asp at position 213. In some embodiments, two, three, four, five, six, seven, or all eight of positions 118, 119, 120, 122, 210, 211, 212, and 213 have a substitution as specified in this paragraph. In some embodiments, a modified CH3 domain polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set.

In some embodiments, a modified CH3 domain polypeptide has at least 70% identity, at least 75% identity, at least 80% identity, at least 85% identity, at least 90% identity, or at least 95% identity to amino acids 114-220 of SEQ ID NO:1, with the proviso that the percent identity does not include the set of positions 118, 119, 120, 122, 210, 211, 212, and 213.

### CH2 Transferrin Receptor-Binding Polypeptides

In some embodiments, the domain that is modified is a human Ig CH2 domain, such as an IgG CH2 domain. The CH2 domain can be of any IgG subtype, i.e., from IgG1, IgG2, IgG3, or IgG4. In the context of IgG antibodies, a CH2 domain refers to the segment of amino acids from about position 231 to about position 340 as numbered according to the EU numbering scheme. The positions in the CH2 domain for purposes of identifying the corresponding set of amino acid positions for transferrin receptor-binding are determined with reference to SEQ ID NO:2 or determined with reference to amino acids 4-113 of SEQ ID NO:1. Substitutions are also determined with reference to SEQ ID NO:1, i.e., an amino acid is considered to be a substitution relative to the amino acid at the corresponding position in SEQ ID NO:1. SEQ ID NO:1 includes a partial hinge region sequence, PCP, as amino acids 1-3. The three residues are not part of the Fc region; however, the numbering of the positions in the CH2 domain with reference to SEQ ID NO:1 includes the first three amino acids.

As indicated above, sets of residues of a CH2 domain that can be modified are numbered herein with reference to SEQ ID NO:1. Any CH2 domain, e.g., an IgG1, IgG2, IgG3, or IgG4 CH2 domain, may have modifications, e.g., amino acid substitutions, in one or more sets of residues that correspond to residues at the noted positions in SEQ ID NO:1. The positions of each of the IgG2, IgG3, and IgG4 sequences that correspond to any given position of SEQ ID NO:1 can be readily determined.

In one embodiment, a modified CH2 domain polypeptide that specifically binds transferrin receptor binds to an epitope in the apical domain of the transferrin receptor. The human transferrin receptor apical domain sequence is set forth in SEQ ID NO:4, which corresponds to amino acids 198-378 of the human transferrin receptor-1 uniprotein sequence P02786. The modified CH2 domain polypeptide may bind to the transferrin receptor without blocking or otherwise inhibiting binding of transferrin to the receptor. In some embodiments, binding of transferrin to TfR is not substantially inhibited. In some embodiments, binding of transferrin to TfR is inhibited by less than about 50% (e.g., less than about 45%, 40%, 35%, 30%, 25%, 20%, 15%, 10%, or 5%). In some embodiments, binding of transferrin to TfR is inhibited by less than about 20% (e.g., less than about 19%, 18%, 17%, 16%, 15%, 14%, 13%, 12%, 11%, 10%, 9%, 8%, 7%, 6%, 5%, 4%, 3%, 2%, or 1%).

CH2 Transferrin Receptor Binding Set (iii): 47, 49, 56, 58, 59, 60, 61, 62, and 63

In some embodiments, a modified CH2 domain polypeptide comprises at least three or at least four, and typically five, six, seven, eight, or nine substitutions in a set of amino acid positions comprising 47, 49, 56, 58, 59, 60, 61, 62, and 63 (set iii). In some embodiments, the modified CH2 domain polypeptide comprises Glu at position 60 and/or Trp at position 61. In some embodiments, the modified CH2 domain polypeptide comprises at least one substitution at a position as follows: Glu, Gly, Gln, Ser, Ala, Asn, Tyr, or Trp at position 47; Ile, Val, Asp, Glu, Thr, Ala, or Tyr at position 49; Asp, Pro, Met, Leu, Ala, Asn, or Phe at position 56; Arg, Ser, Ala, or Gly at position 58; Tyr, Trp, Arg, or Val at position 59; Glu at position 60; Trp or Tyr at position 61; Gln, Tyr, His, Ile, Phe, Val, or Asp at position 62; or Leu, Trp, Arg, Asn, Tyr, or Val at position 63. In some embodiments, two, three, four, five, six, seven, eight, or all nine of positions 47, 49, 56, 58, 59, 60, 61, 62, and 63 have a substitution as specified in this paragraph. In some embodiments, a modified CH2 domain polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set.

In some embodiments, a modified CH2 domain polypeptide has at least 70% identity, at least 75% identity, at least 80% identity, at least 85% identity, at least 90% identity, or at least 95% identity to amino acids 4-113 of SEQ ID NO:1, with the proviso that the percent identity does not include the set of positions 47, 49, 56, 58, 59, 60, 61, 62, and 63.

CH2 Transferrin Receptor Binding Set (iv): 39, 40, 41, 42, 43, 44, 68, 70, 71, and 72

In some embodiments, a modified CH2 domain polypeptide comprises at least three or at least four, and typically five, six, seven, eight, nine, or ten substitutions in a set of amino acid positions comprising 39, 40, 41, 42, 43, 44, 68, 70, 71, and 72 (set iv). In some embodiments, the modified CH2 domain polypeptide comprises Pro at position 43, Glu at position 68, and/or Tyr at position 70. In some embodiments, the modified CH2 domain polypeptide comprises at least one substitution at a position as follows: Pro, Phe, Ala, Met, or Asp at position 39; Gln, Pro, Arg, Lys, Ala, Ile, Leu, Glu, Asp, or Tyr at position 40; Thr, Ser, Gly, Met, Val, Phe, Trp, or Leu at position 41; Pro, Val, Ala, Thr, or Asp at position 42; Pro, Val, or Phe at position 43; Trp, Gln, Thr, or Glu at position 44; Glu, Val, Thr, Leu, or Trp at position 68; Tyr, His, Val, or Asp at position 70; Thr, His, Gln, Arg, Asn, or Val at position 71; or Tyr, Asn, Asp, Ser, or Pro at position 72. In some embodiments, two, three, four, five, six, seven, eight, nine, or all ten of positions 39, 40, 41, 42, 43, 44, 68, 70, 71, and 72 have a substitution as specified in this paragraph. In some embodiments, a modified CH2 domain polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set.

In some embodiments, a modified CH2 domain polypeptide has at least 70% identity, at least 75% identity, at least 80% identity, at least 85% identity, at least 90% identity, or at least 95% identity to amino acids 4-113 of SEQ ID NO:1, with the proviso that the percent identity does not include the set of positions 39, 40, 41, 42, 43, 44, 68, 70, 71, and 72.

CH2 Transferrin Receptor Binding Set (v): 41, 42, 43, 44, 45, 65, 66, 67, 69, and 73

In some embodiments, a modified CH2 domain polypeptide comprises at least three or at least four, and typically five, six, seven, eight, nine, or ten substitutions in a set of amino acid positions comprising 41, 42, 43, 44, 45, 65, 66, 67, 69, and 73 (set v). In some embodiments, the modified CH2 domain polypeptide comprises at least one substitution at a position as follows: Val or Asp at position 41; Pro, Met, or Asp at position 42; Pro or Trp at position 43; Arg, Trp, Glu, or Thr at position 44; Met, Tyr, or Trp at position 45; Leu or Trp at position 65; Thr, Val, Ile, or Lys at position 66; Ser, Lys, Ala, or Leu at position 67; His, Leu, or Pro at position 69; or Val or Trp at position 73. In some embodiments, two, three, four, five, six, seven, eight, nine, or all ten of positions 41, 42, 43, 44, 45, 65, 66, 67, 69, and 73 have a substitution as specified in this paragraph. In some embodiments, a modified CH2 domain polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set.

In some embodiments, a modified CH2 domain polypeptide has at least 70% identity, at least 75% identity, at least 80% identity, at least 85% identity, at least 90% identity, or at least 95% identity to amino acids 4-113 of SEQ ID NO:1, with the proviso that the percent identity does not include the set of positions 41, 42, 43, 44, 45, 65, 66, 67, 69, and 73.

CH2 Transferrin Receptor Binding Set (vi): 45, 47, 49, 95, 97, 99, 102, 103, and 104

In some embodiments, a modified CH2 domain polypeptide comprises at least three or at least four, and typically five, six, seven, eight, or nine substitutions in a set of amino acid positions comprising 45, 47, 49, 95, 97, 99, 102, 103, and 104 (set vi). In some embodiments, the modified CH2 domain polypeptide comprises Trp at position 103. In some embodiments, the modified CH2 domain polypeptide comprises at least one substitution at a position as follows: Trp, Val, Ile, or Ala at position 45; Trp or Gly at position 47; Tyr, Arg, or Glu at position 49; Ser, Arg, or Gln at position 95; Val, Ser, or Phe at position 97; Ile, Ser, or Trp at position 99; Trp, Thr, Ser, Arg, or Asp at position 102; Trp at position 103; or Ser, Lys, Arg, or Val at position 104. In some embodiments, two, three, four, five, six, seven, eight, or all nine of positions 45, 47, 49, 95, 97, 99, 102, 103, and 104 have a substitution as specified in this paragraph. In some embodiments, a modified CH2 domain polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set.

In some embodiments, a modified CH2 domain polypeptide has at least 70% identity, at least 75% identity, at least 80% identity, at least 85% identity, at least 90% identity, or at least 95% identity to amino acids 4-113 of SEQ ID NO:1, with the proviso that the percent identity does not include the set of positions 45, 47, 49, 95, 97, 99, 102, 103, and 104.

### V. Additional Mutations in an Fc Region

A polypeptide (e.g., that is modified to bind a transferrin receptor and can initiate transport across the BBB) that is linked to an agent for use in methods of the present invention may also comprise additional mutations, e.g., to increase serum stability, to modulate effector function, to influence glycosylation, to reduce immunogenicity in humans, and/or to provide for knob and hole heterodimerization of the polypeptide.

In some embodiments, a polypeptide has an amino acid sequence identity of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% to a corresponding wild-type Fc region (e.g., a human IgG1, IgG2, IgG3, or IgG4 Fc region).

A polypeptide may also have other mutations introduced outside of the specified sets of amino acids, e.g., to influence glycosylation, to increase serum half-life or, for CH3 domains, to provide for knob and hole heterodimerization of polypeptides that comprise the modified CH3 domain. Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine). Such additional mutations are at a position in the polypeptide that does not have a negative effect on binding of a modified CH3 or CH2 domain to the transferrin receptor.

In one illustrative embodiment of a knob and hole approach for dimerization, a position corresponding to position 139 of SEQ ID NO:1 of a first Fc polypeptide subunit to be dimerized has a tryptophan in place of a native threonine and a second Fc polypeptide subunit of the dimer has a valine at a position corresponding to position 180 of SEQ ID NO:1 in place of the native tyrosine. The second subunit of the Fc polypeptide may further comprise a substitution in which the native threonine at the position corresponding to position 139 of SEQ ID NO:1 is substituted with a serine and a native leucine at the position corresponding to position 141 of SEQ ID NO:1 is substituted with an alanine.

A polypeptide may also be engineered to contain other modifications for heterodimerization, e.g., electrostatic engineering of contact residues within a CH3-CH3 interface that are naturally charged or hydrophobic patch modifications.

In some embodiments, modifications to enhance serum half-life may be introduced. For example, in some embodiments, an Fc region comprises a CH2 domain comprising a Tyr at a position corresponding to position 25 of SEQ ID NO:1, Thr at a position corresponding to 27 of SEQ ID NO:1, and Glu at a position corresponding to position 29 of SEQ ID NO:1.

In some embodiments, a mutation, e.g., a substitution, is introduced at one or more of positions 17-30, 52-57, 80-90, 156-163, and 201-208 as determined with reference to SEQ ID NO:1. In some embodiments, one or more mutations are introduced at positions 24, 25, 27, 28, 29, 80, 81, 82, 84, 85, 87, 158, 159, 160, 162, 201, 206, 207, or 209 as determined with reference to SEQ ID NO:1. In some embodiments, mutations are introduced into one, two, or three of positions 25, 27, and 29 as determined with reference to SEQ ID NO:1. In some embodiments, the mutations are M25Y, S27T, and T29E as numbered with reference to SEQ ID NO:1. In some embodiments, a polypeptide as described herein further comprises mutations M25Y, S27T, and T29E. In some embodiments, mutations are introduced into one or two of positions 201 and 207 as determined with reference to SEQ ID NO:1. In some embodiments, the mutations are M201L and N207S as numbered with reference to SEQ ID NO:1. In some embodiments, a polypeptide as described herein further comprises mutation N207S with or without M201L. In some embodiments, a polypeptide as described herein comprises a substitution at one, two or all three of positions T80, E153, and N207 as numbered with reference to SEQ ID NO:1. In some embodiments, the mutations are T80Q and N207A. In some embodiments, a polypeptide as described herein comprises mutations T80A, E153A, and N207A. In some embodiments, a polypeptide as described herein comprises substitutions at positions T23 and M201 as numbered with reference to SEQ ID NO:1. In some embodiments, a polypeptide as described herein comprises mutations T23Q and M201L. In some embodiments, a polypeptide as described herein comprises substitutions at positions M201 and N207 as numbered with reference to SEQ ID NO:1. In some embodiments, a polypeptide as described herein comprises substitutions M201L and N207S. In some embodiments, a polypeptide as described herein comprises an N207S or N207A substitution.

Fc Effector Functions

In some embodiments, an Fc region (e.g., comprising a modified CH2 or CH3 domain) has an an effector function, i.e., the Fc region has the ability to induce certain biological functions upon binding to an Fc receptor expressed on an effector cell that mediates the effector function. Effector cells include, but are not limited to, monocytes, macrophages, neutrophils, dendritic cells, eosinophils, mast cells, platelets, B cells, large granular lymphocytes, Langerhans' cells, natural killer (NK) cells, and cytotoxic T cells.

Examples of antibody effector functions include, but are not limited to, C1q binding and complement dependent cytotoxicity (CDC), Fc receptor binding, antibody-dependent cell-mediated cytotoxicity (ADCC), antibody-dependent cell-mediated phagocytosis (ADCP), down-regulation of cell surface receptors (e.g., B cell receptor), and B-cell activation. Effector functions may vary with the antibody class. For example, native human IgG1 and IgG3 antibodies can elicit ADCC and CDC activities upon binding to an appropriate Fc receptor present on an immune system cell; and native human IgG1, IgG2, IgG3, and IgG4 can elicit ADCP functions upon binding to the appropriate Fc receptor present on an immune cell.

In some embodiments, a polypeptide as described herein may include additional modifications that reduce effector function. Alternatively, in some embodiments, a polypeptide (e.g., comprising a modified CH2 or CH3 domain) may include additional modifications that enhance effector function.

Illustrative Fc polypeptide mutations that modulate an effector function include, but are not limited to, substitutions in a CH2 domain, e.g., at positions corresponding to positions 7 and 8 of SEQ ID NO:1. In some embodiments, the substitutions in a modified CH2 domain comprise Ala at positions 7 and 8 of SEQ ID NO:1. In some embodiments, the substitutions in a modified CH2 domain comprise Ala at positions 7 and 8 and Gly at position 102 of SEQ ID NO:1.

Additional Fc polypeptide mutations that modulate an effector function include, but are not limited to, one or more substitutions at positions 238, 265, 269, 270, 297, 327 and 329 (EU numbering scheme, which correspond to positions 11, 38, 42, 43, 70, 100, and 102 as numbered with reference to SEQ ID NO:1). Illustrative substitutions (as numbered with EU numbering scheme), include the following: position 329 may have a mutation in which proline is substituted with a glycine or arginine or an amino acid residue large enough to destroy the Fc/Fcγ receptor interface that is formed between proline 329 of the Fc and tryptophan residues Trp 87 and Trp 110 of FcγRIII. Additional illustrative substitutions include S228P, E233P, L235E, N297A, N297D, and P331S. Multiple substitutions may also be present, e.g., L234A and L235A of a human IgG1 Fc region; L234A, L235A, and P329G of a human IgG1 Fc region; S228P and L235E of a human IgG4 Fc region; L234A and G237A of a human IgG1 Fc region; L234A, L235A, and G237A of a human IgG1 Fc region; V234A and G237A of a human IgG2 Fc region; L235A, G237A, and E318A of a human IgG4 Fc region; and S228P and L236E of a human IgG4 Fc region. In some embodiments, a polypeptide may have one or more amino acid substitutions that modulate ADCC, e.g., substitutions at positions 298, 333, and/or 334 of the Fc region, according to the EU numbering scheme.

In some embodiments, a polypeptide as described herein may have one or more amino acid substitutions that increase or decrease ADCC or may have mutations that alter C1q binding and/or CDC.

Illustrative Polypeptides Comprising Additional Mutations

A polypeptide may comprise additional mutations including a knob mutation (e.g., T139W as numbered with reference to SEQ ID NO:1), hole mutations (e.g., T139S, L141A, and Y180V as numbered with reference to SEQ ID NO:1), mutations that modulate effector function (e.g., L7A, L8A, and/or P102G (e.g., L7A and L8A) as numbered with reference to SEQ ID NO:1), and/or mutations that increase serum stability (e.g., (i) M25Y, S27T, and T29E as numbered with reference to SEQ ID NO:1, or (ii) N207S with or without M201L as numbered with reference to SEQ ID NO:1).

### VI. Examples

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results. Efforts have been made to ensure accuracy with respect to numbers used (e.g., amounts, temperatures, etc.), but some experimental error and deviation may be present. The practice of the present invention will employ, unless otherwise indicated, conventional methods of protein chemistry, biochemistry, recombinant DNA techniques and pharmacology, within the skill of the art. Such techniques are explained fully in the literature. Additionally, it should be apparent to one of skill in the art that the methods for engineering as applied to certain libraries can also be applied to other libraries described herein.

### Example 1. Pharmacokinetic/Pharmacodynamic Characterization of CH3C Variants

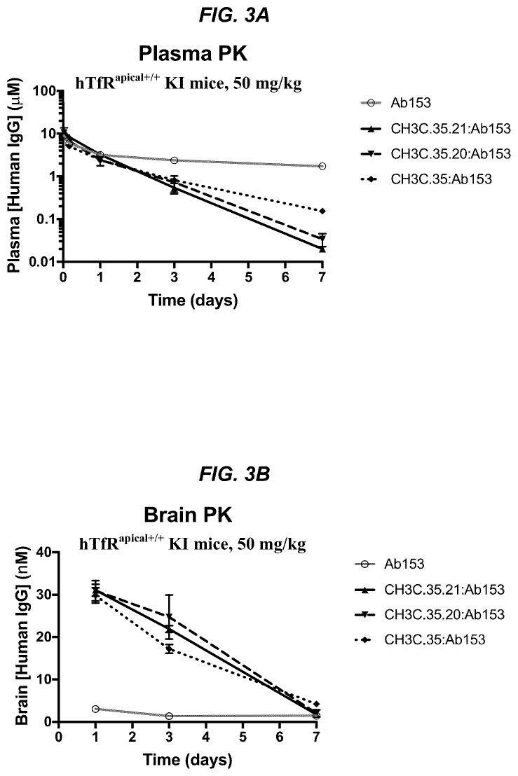

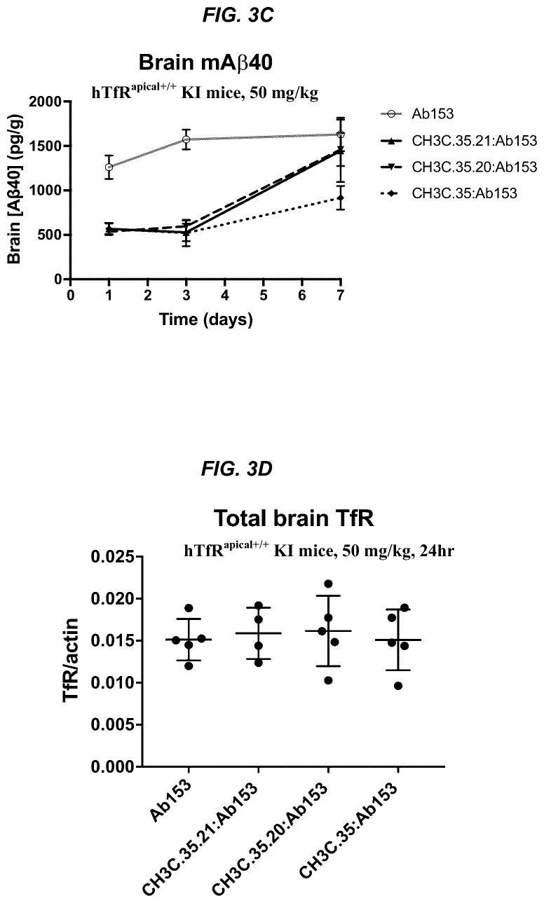

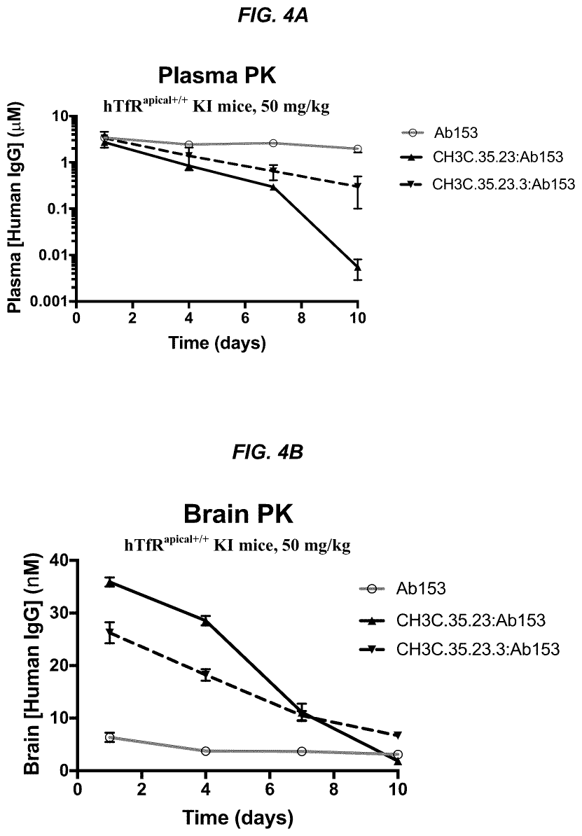

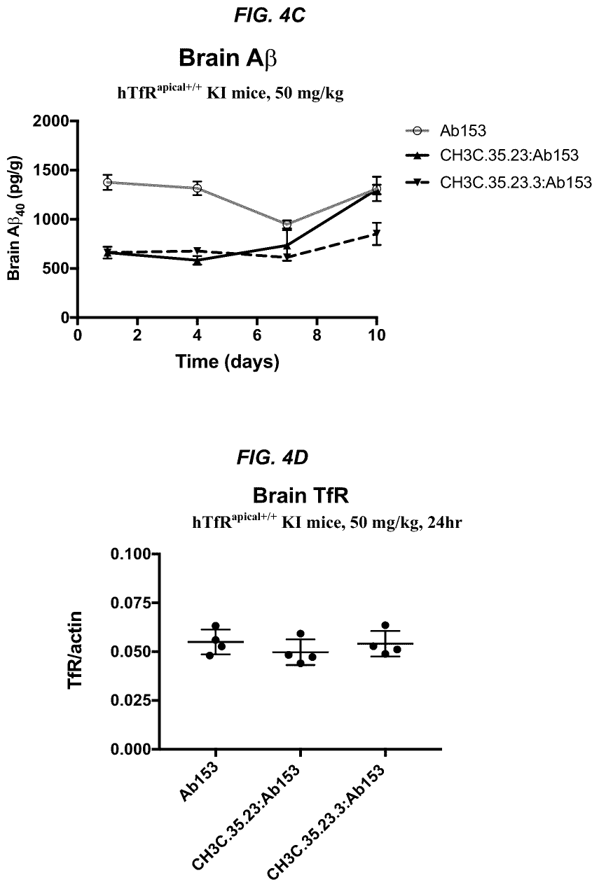

This example describes pharmacokinetic/pharmacodynamic (PK/PD) characterization of CH3C variant polypeptides in mouse plasma and brain tissue. In particular, this example shows that an anti-BACE1 agent exhibited lower Cₘₐₓ values in the brain, but higher brain concentrations over time and more Abeta inhibition over time, when linked to a polypeptide with a weaker affinity for TfR as compared to a polypeptide with a stronger affinity for TfR (see, e.g., FIGS. 3B, 3C, 4B, and 4C).

### Pharmacokinetics of CH3C Variants in Wild-Type Mouse Plasma

Pharmacokinetics (PK) were tested for several CH3C variants in wild-type mice to demonstrate in vivo stability in a model lacking TfR-mediated clearance, as the polypeptide-Fab fusions bind only human TfR and not murine TfR. The study design is shown in Table 3 below. 6-8 week-old C57B16 mice were intravenously dosed and in-life bleeds were taken via submandibular-bleeds, at time points as indicated in Table 3. Blood was collected in EDTA plasma tubes, spun at 14,000 rpm for 5 minutes, and then plasma was isolated for subsequent analysis.

TABLE 3
PK study design

| Group | Polypeptide | Time points | N | Dose (IV) |
|---|---|---|---|---|
| 1A/1B | Ab122 | A = 30 min, 24 h, 4 d | A = 2 | 12.3 mg/kg |
| | | B = 4 h, 2 d, 7 d | B = 3 | |
| 2A/2B | Ab153 | A = 30 min, 24 h, 4 d | A = 2 | 11.4 mg/kg |
| | | B = 4 h, 2 d, 7 d | B = 3 | |
| 3A/3B | CH3C.35.163 | A = 30 min, 24 h, 4 d | A = 2 | 11.4 mg/kg |
| | mono (Ab153 | B = 4 h, 2 d, 7 d | B = 3 | |
| | fusion) | | | |
| 4A/4B | CH3C.3.2-19 | A = 30 min, 24 h, 4 d | A = 2 | 11.0 mg/kg |
| | (Ab153 fusion) | B = 4 h, 2 d, 7 d | B = 3 | |
| 5A/5B | CH3C.3.2-5 | A = 30 min, 24 h, 4 d | A = 2 | 10.5 mg/kg |
| | (Ab153 fusion) | B = 4 h, 2 d, 7 d | B = 3 | |
| 6A/6B | CH3C.3.2-1 | A = 30 min, 24 h, 4 d | A = 2 | 10.0 mg/kg |
| | (Ab153 fusion) | B = 4 h, 2 d, 7 d | B = 3 | |

Ab122 served as an anti-RSV control that has normal PK in mice. Ab153 served as an anti-BACE1 control that has normal PK in mice. The Fab arms of Ab153 were fused to the polypeptides in this study.

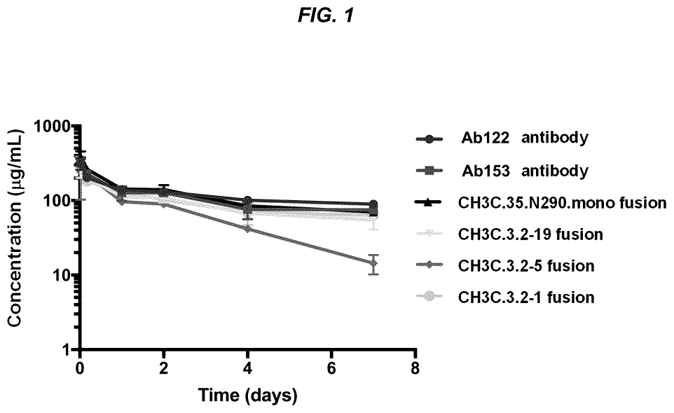

Polypeptide concentrations in mouse plasma were quantified using a generic human IgG assay (MSD® human IgG kit #K150JLD-4) following the manufacturer's instructions. Briefly, precoated plates were blocked for 30 minutes with MSD® Blocker A. Plasma samples were diluted 1:2,500 using a Hamilton® NIMBUS liquid handler and added in duplicate to the blocked plates. Dosing solutions were also analyzed on the same plate to confirm the correct dosage. The standard curve, 0.78-200 ng/mL IgG, was fit using a four-parameter logistic regression. FIG. 1 and Table 4 show the analysis of these data. All of the CH3C polypeptide variants had clearance and half-life values comparable to the standard Ab122, except for CH3C.3.2-5, which had substantially faster clearance and a shorter half-life. Interestingly, this variant was a point mutant of CH3C.3.2-19 (N163D), the latter of which had a normal PK profile.

TABLE 4
PK parameters for CH3C polypeptide-Fab fusions

| | Clearance | Half-life |
|---|---|---|
| Polypeptide | (mg/day/kg) | (days) |
| Ab122 | 6.12 | 9.12 |
| Ab153 | 9.11 | 4.74 |
| CH3C.35.N163 mono (Ab153 fusion) | 8.44 | 5.35 |
| CH3C.3.2-19 (Ab153 fusion) | 10.3 | 5.42 |
| CH3C.3.2-5 (Ab153 fusion) | 21.0 | 1.90 |
| CH3C.3.2-1 (Ab153 fusion) | 9.25 | 4.65 |

### PK/PD Evaluation of Monovalent CH3C.35.N163 in Wild-Type Mouse Brain Tissue

Transgenic mice expressing human Tfrc apical domain within the murine Tfrc gene were generated using CRISPR/Cas9 technology. The resulting chimeric TfR was expressed in vivo under the control of the endogenous promoter.

Chimeric hTfR^{apical+/+} heterozygous mice (n=4/group) were intravenously dosed with 42 mg/kg of either Ab153 or monovalent CH3C.35.N163, and wild-type mice (n=3) were dosed intravenously with 50 mg/kg of control human IgG1. Ab153 served as a control that has normal PK in mice. All mice were perfused with PBS 24 hours post-dose. Prior to perfusion, blood was collected in EDTA plasma tubes via cardiac puncture and spun at 14,000 rpm for 5 minutes. Plasma was then isolated for subsequent PK and PD analysis. Brains were extracted after perfusion and hemi-brains were isolated for homogenization in 10× by tissue weight of 1% NP-40 in PBS (for PK) or 5 M GuHCl (for PD).

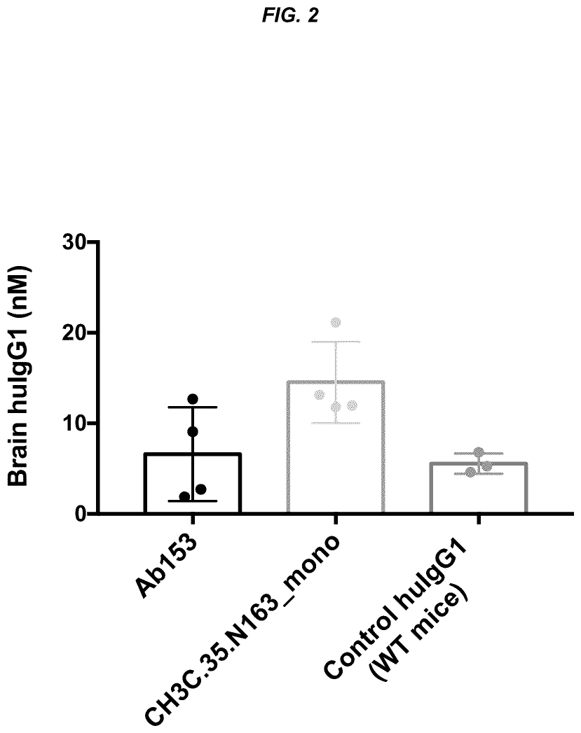

FIG. 2 shows the results of the brain PK study. Uptake was greater in the monovalent CH3C.35.N163 group than the Ab153 and control human IgG1 groups.

Brain and Plasma PKPD of Polypeptide-Fab Fusions in hTfR^{apical+/+} Mice: CH3C.35.21, CH3C.35.20, CH3C.35, CH3C.35.23, CH3C.35.23.3

To evaluate the impact of TfR binding affinity for PK and brain uptake, anti-BACE1 Ab153 and engineered TfR-binding polypeptide fusions (CH3C.35.21:Ab153, CH3C.35.20:Ab153, CH3C.35:Ab153 fusions) were generated that differed in their binding affinity to apical human TfR as measured by Biacore. The binding affinities of CH3C.35.21:Ab153, CH3C.35.20:Ab153, CH3C.35:Ab153 fusions to human TfR are 100 nM, 170 nM and 620 nM, respectively. hTfR^{apical+/+} knock-in mice were systemically administered either Ab153 or the polypeptide-Fab fusions at 50 mg/kg, and plasma PK and brain PKPD was evaluated at 1, 3, and 7 days post-dose. Brain and plasma PKPD analysis was conducted as described in the previous section. Due to expression of TfR on peripheral tissues, CH3C.35.21:Ab153, CH3C.35.20:Ab153, and CH3C.35:Ab153 fusions exhibited faster clearance in plasma as compared to Ab153 alone, consistent with target-mediated clearance and indicative of in vivo TfR binding (FIG. 3A). Impressively, brain concentrations of CH3C.35.21:Ab153, CH3C.35.20:Ab153, and CH3C.35:Ab153 fusions were significantly increased compared to Ab153, achieving a maximum brain concentration of more than 30 nM at 1 day post-dose, compared to only about 3 nM for Ab153 at this same time point (FIG. 3B). The increase in brain exposure of CH3C.35.21:Ab153, CH3C.35.20:Ab153, and CH3C.35:Ab153 fusions resulted in about 55-60% lower endogenous mouse Aβ levels in brains of mice compared to Aβ levels in mice dosed with Ab153 (FIG. 3C). The lower brain Aβ levels were sustained while concentrations of CH3C.35.21:Ab153, CH3C.35.20:Ab153, and CH3C.35:Ab153 fusions remained elevated in brain, and returned to levels similar to Ab153 treated mice at when exposure was reduced by day 7. The reduction in brain exposure over time correlated with a reduction in peripheral exposure of CH3C.35.21:Ab153, CH3C.35.20:Ab153, and CH3C.35:Ab153 fusions, providing a clear PK/PD relationship in vivo (compare FIGS. 3A and 3C). Additionally, total brain TfR levels were comparable for Ab153-treated and polypeptide-Fab fusion-treated mice after this single high dose, indicating no significant impact of increased brain exposure of the polypeptide-Fab fusions to TfR expression in brain (FIG. 3D).

To further evaluate the relationship between PK and brain uptake with a wider affinity range of engineered TfR-binding polypeptide-Fab fusions, additional fusions with a wider affinity range for hTfR binding was generated. The binding affinities of CH3C.35.23:Ab153 and CH3C.35.23.3:Ab153 fusions to human TfR are 420 nM and 1440 nM, respectively. hTfRapical^{+/+} knock-in mice were dosed as described above. Plasma PK and brain PKPD were evaluated at 1, 4, 7, and 10 days post-dose. Peripheral PK of the polypeptide-Fab fusions were hTfR affinity-dependent, where the higher affinity CH3C.35.23:Ab153 fusion exhibited faster clearance compared to the much lower affinity CH3C.35.23.3:Ab153 fusion (FIG. 4A). Both CH3C.35.23:Ab153 and CH3C.35.23.3:Ab153 fusions had significantly greater brain exposure than compared to Ab153 alone, with CH3C.35.23:Ab153 achieving about 36 nM in brain at 1 day post-dose (FIG. 4B). Despite similar plasma concentrations, this maximum brain uptake of CH3C.35.23.3:Ab153 fusion was lower than that of CH3.35.23:Ab153 fusion, likely due to the about 3.5-fold lower affinity of the latter fusion for hTfR. Interestingly, because the lower affinity fusion provided a more sustained peripheral exposure by day 10, its brain exposure was also higher than that of the higher affinity CH3C.35.23:Ab153 fusion. This illustrates a trade-off of lower brain Cₘₐₓ but more sustained PK over time for lower affinity engineered TfR-binding polypeptide-Fab Fusions. Significantly lower concentrations of Aβ40 was observed in brains of mice dosed with the anti-BACE1 polypeptide fusions compared to anti-BACE1 alone (FIG. 4C). This duration of Aβ40 reduction was consistent with levels of huIgG1 exposure in brain over time (FIG. 4B). Impressively, mice dosed with CH3C.35:Ab153 fusion exhibited a prolonged brain Aβ40 reduction out to 7-10 days after a single dose. Total brain TfR levels were comparable between mice dosed with Ab153 versus CH3C.35:Ab153 fusion at 1 day post-dose (FIG. 4D). Together these data demonstrate that engineered TfR-binding polypeptide fusion can increase brain exposure of anti-BACE1 to significantly reduce brain Aβ40 after a single dose.

### Example 2. Selection of TfR-Binding Polypeptide Affinity

This example describes the relationship between the affinity of a TfR-binding polypeptide for a transferrin receptor (TfR) and the resulting brain exposure to a therapeutic agent that is linked to the TfR-binding polypeptide.

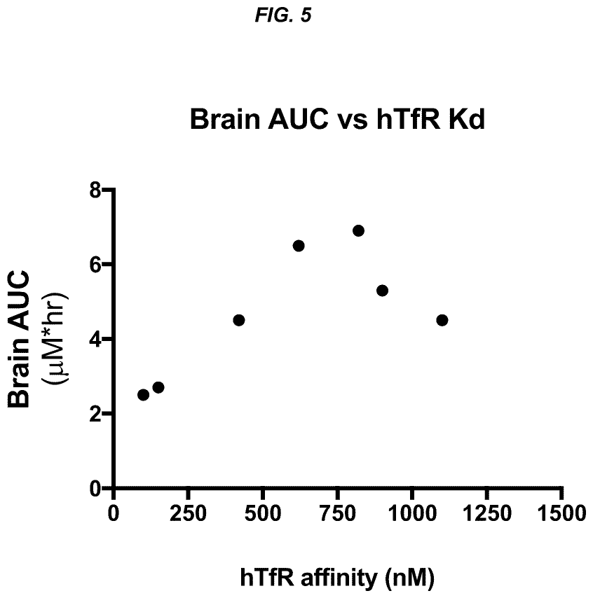

FIG. 5 shows that brain exposure to a therapeutic agent (as assessed by determining the area under the curve (AUC) of brain concentration vs. time) was greatest when the therapeutic agent was linked to a polypeptide that had a relatively lower affinity for TfR. In particular, brain exposure was substantially prolonged when the therapeutic agent was linked to a polypeptide that had an affinity for TfR that was weaker than about 250 nM.

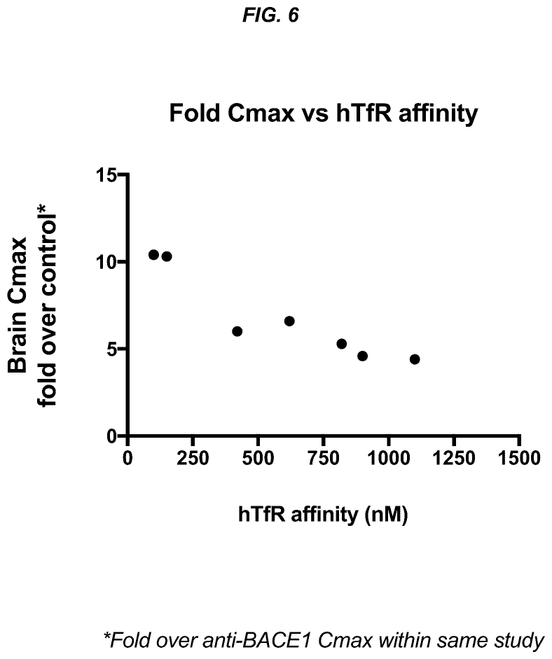

As shown in FIG. 6, lower maximum concentration (Cₘₐₓ) in the brain was observed when a therapeutic agent was linked to a polypeptide that had a relatively weaker affinity for a TfR. In particular, brain Cₘx values were significantly lower when the TfR-binding polypeptide had an affinity that was weaker than about 250 nM.

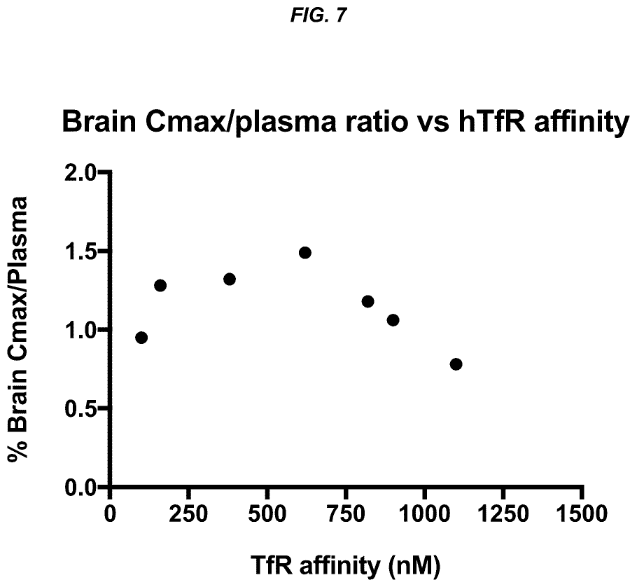

FIG. 7 shows the ratio of brain Cₘₐₓ to plasma concentration of a therapeutic agent when linked to polypeptides having a range of affinities for TfR.

### Methods

Generation of hTfR^{apical+/+} KI

Methods for generating knock-in/knock-out mice have been published in the literature and are well known to those with skill in the art. In summary, hTfR^{apical+/+} KI mice were generated using CRISPR/Cas9 technology to express human Tfrc apical domain within the murine Tfrc gene; the resulting chimeric TfR was expressed in vivo under the control of the endogenous promoter. As described in International Patent Application No. PCT/US2018/018302, which is incorporated by reference in its entirety herein, C57B16 mice were used to generate a knock-in of the human apical TfR mouse line via pronuclear microinjection into single cell embryos, followed by embryo transfer to pseudo pregnant females. Specifically, Cas9, single guide RNAs and a donor DNA were introduced into the embryos. The donor DNA comprised a human apical domain coding sequence that had been codon optimized for expression in mouse. The apical domain coding sequence was flanked with a left and a right homology arm. The donor sequence was designed such that the apical domain was inserted after the fourth mouse exon, and was immediately flanked at the 3′ end by the ninth mouse exon. A founder male from the progeny of the female that received the embryos was bred to wild-type females to generate F1 heterozygous mice. Homozygous mice were subsequently generated from breeding of F1 generation heterozygous mice.

### Mouse PKPD

For PK/PD evaluation, hTfR^{apical+/+} KI mice were systemically dosed one time via tail vein injection at 50 mg/kg. Prior to perfusion, blood was collected in EDTA plasma tubes via cardiac puncture and spun at 14,000 rpm for 5 minutes. Plasma was then isolated for subsequent PK/PD analysis. Brains were extracted after perfusion and hemi-brains were isolated for homogenization in 10× by tissue weight of 1% NP-40 in PBS (for PK) or 5 M GuHCl (for PD).

Antibody concentrations in mouse plasma and brain lysates were quantified using a generic human IgG assay (MSD human IgG kit #K150JLD) following the manufacturer's instructions. Briefly, pre-coated plates were blocked for 30 minutes with MSD Blocker A. Plasma samples were diluted 1:10,000 using a Hamilton Nimbus liquid handler and added in duplicate to the blocked plates. Brain samples were homogenized in 1% NP-40 lysis buffer and lysates diluted 1:10 for PK analysis. Dosing solutions were also analyzed on the same plate to confirm the correct dosage. The standard curve, 0.78-200 ng/mL IgG, was fit using a four-parameter logistic regression.

### Example 3. Binding Characterization of CH3C Variants Using Biacore™

The affinity of clone variants for recombinant TfR apical domain was determined by surface plasmon resonance using a Biacore™ T200 instrument. Biacore™ Series S CM5 sensor chips were immobilized with anti-human Fab (human Fab capture kit from GE Healthcare). 5 μg/mL of polypeptide-Fab fusion was captured for 1 minute on each flow cell and serial 3-fold dilutions of human or cyano apical domain were injected at a flow rate of 30 μL/min at room temperature. Each sample was analyzed with a 45-second association and a 3-minute dissociation. After each injection, the chip was regenerated using 10 mM glycine-HCl (pH 2.1). Binding response was corrected by subtracting the RU from a flow cell capturing an irrelevant IgG at similar density. Steady-state affinities were obtained by fitting the response at equilibrium against the concentration using Biacore™ T200 Evaluation Software v3.1.

To determine the affinity of clone variants for recombinant TfR ectodomain (ECD), Biacore™ Series S CM5 sensor chips were immobilized with streptavidin. Biotinylated human or cyano TfR ECD was captured for 1 minute on each flow cell and serial 3-fold dilutions of clone variants were injected at a flow rate of 30 μL/min at room temperature. Each sample was analyzed with a 45-second association and a 3-minute dissociation. The binding response was corrected by subtracting the RU from a flow cell without TfR ECD at a similar density. Steady-state affinities were obtained by fitting the response at equilibrium against the concentration using Biacore™ T200 Evaluation Software v3.1.

The binding affinities are summarized in Table 5. Affinities were obtained by steady-state fitting.

TABLE 5
Binding affinities for additional CH3C variants

| | Human TfR | Cyno TfR | Human apical | Cyno apical |
|---|---|---|---|---|
| Clone | (μM) | (μM) | TfR (μM) | TfR (μM) |
| CH3C.35.19.mono | 0.4 | 5.9 | 0.37 | 5.6 |
| CH3C.35.20.mono | 0.25 | 6.7 | 0.17 | 8 |
| CH3C.35.21.mono | 0.1 | 2.1 | 0.12 | 2.2 |
| CH3C.35.24.mono | 0.29 | 3.3 | 0.23 | 3 |
| CH3C.35.21.11.mono | 0.24 | 4 | 0.13 | 2.2 |
| CH3C.35.21.16.mono | 0.18 | 1.8 | 0.12 | 1.9 |
| CH3C.35.21.17.mono | 0.3 | 2.9 | 0.13 | 2.6 |
| CH3C.35.mono | 0.61 | >10 | 0.61 | >10 |
| CH3C.35.N153.mono | 0.42 | >10 | 0.95 | >10 |
| CH3C.35.bi | 0.22 | >2 | not tested | not tested |
| CH3C.35.N153.bi | 0.37 | 3.3 | not tested | not tested |
| CH3C.3.2-19.bi | 5.2 | 5.6 | not tested | not tested |
| CH3C.35.19.bi | 0.074 | 1.5 | not tested | not tested |
| CH3C.35.20.bi | 0.054 | 1.7 | not tested | not tested |
| CH3C.35.21.bi | 0.049 | 0.7 | not tested | not tested |
| CH3C.35.24.bi | 0.061 | 0.65 | not tested | not tested |

Additional CH3C variants CH3C.35.20.1.1, CH3C.35.23.2.1, CH3C.35.23.1.1, CH3C.35.S413, CH3C.35.23.3.1, CH3C.35.N390.1, and CH3C.35.23.6.1 were created and their binding affinities to human TfR were measured following the same protocol as previously described. The binding affinities of CH3C.35.20.1.1, CH3C.35.23.2.1, CH3C.35.23.1.1, CH3C.35.S413, CH3C.35.23.3.1, CH3C.35.N390.1, and CH3C.35.23.6.1 are 620 nM, 690 nM, 750 nM, 1700 nM, 1900 nM, 2000 nM, and 2100 nM, respectively.

It is understood that the examples and embodiments described herein are for illustrative purposes only and that various modifications or changes in light thereof will be suggested to persons skilled in the art and are to be included within the spirit and purview of this application and scope of the appended claims. The sequences of the sequence accession numbers cited herein are hereby incorporated by reference.

TABLE 1
CH3C Register Positions and Mutations

| Sequence | Seq. | | | | | | | | | | | | | | | | | | |
|---|---|---|---|---|---|---|---|---|---|---|---|---|---|---|---|---|---|---|---|
| name | group | 157 | 158 | 159 | 160 | 161 | 162 | 163 | 164 | . . . | 186 | 187 | 188 | 189 | 190 | 191 | 192 | 193 | 194 |
| Wild-type | n/a | N | G | Q | P | E | N | N | Y | . . . | D | K | S | R | W | Q | Q | G | N |
| CH3C.1 | | L | G | L | V | W | V | G | Y | . . . | A | K | S | T | W | Q | Q | G | W |
| CH3C.2 | | Y | G | T | V | W | S | H | Y | . . . | S | K | S | E | W | Q | Q | G | Y |
| CH3C.3 | | Y | G | T | E | W | S | Q | Y | . . . | E | K | S | D | W | Q | Q | G | H |
| CH3C.4 | | V | G | T | P | W | A | L | Y | . . . | L | K | S | E | W | Q | Q | G | W |
| CH3C.17 | 2 | Y | G | T | V | W | S | K | Y | . . . | S | K | S | E | W | Q | Q | G | F |
| CH3C.18 | 1 | L | G | H | V | W | A | V | Y | . . . | P | K | S | T | W | Q | Q | G | W |
| CH3C.21 | 1 | L | G | L | V | W | V | G | Y | . . . | P | K | S | T | W | Q | Q | G | W |
| CH3C.25 | 1 | M | G | H | V | W | V | G | Y | . . . | D | K | S | T | W | Q | Q | G | W |
| CH3C.34 | 1 | L | G | L | V | W | V | F | S | . . . | P | K | S | T | W | Q | Q | G | W |
| CH3C.35 | 2 | Y | G | T | E | W | S | S | Y | . . . | T | K | S | E | W | Q | Q | G | F |
| CH3C.44 | 2 | Y | G | T | E | W | S | N | Y | . . . | S | K | S | E | W | Q | Q | G | F |
| CH3C.51 | 1/2 | L | G | H | V | W | V | G | Y | . . . | S | K | S | E | W | Q | Q | G | W |
| CH3C.3.1-3 | 1 | L | G | H | V | W | V | A | T | . . . | P | K | S | T | W | Q | Q | G | W |
| CH3C.3.1-9 | 1 | L | G | P | V | W | V | H | T | . . . | P | K | S | T | W | Q | Q | G | W |
| CH3C.3.2-5 | 1 | L | G | H | V | W | V | D | Q | . . . | P | K | S | T | W | Q | Q | G | W |
| CH3C.3.2-19 | 1 | L | G | H | V | W | V | N | Q | . . . | P | K | S | T | W | Q | Q | G | W |
| CH3C.3.2-1 | 1 | L | G | H | V | W | V | N | F | . . . | P | K | S | T | W | Q | Q | G | W |
| CH3C.3.4-1 | | W | G | F | V | W | S | T | Y | | P | K | S | N | W | Q | Q | G | F |
| CH3C.3.4-19 | | W | G | H | V | W | S | T | Y | | P | K | S | N | W | Q | Q | G | Y |
| CH3C.3.2-3 | | L | G | H | V | W | V | E | Q | | P | K | S | T | W | Q | Q | G | W |
| CH3C.3.2-14 | | L | G | H | V | W | V | G | V | | P | K | S | T | W | Q | Q | G | W |
| CH3C.3.2-24 | | L | G | H | V | W | V | H | T | | P | K | S | T | W | Q | Q | G | W |
| CH3C.3.4-26 | | W | G | T | V | W | G | T | Y | | P | K | S | N | W | Q | Q | G | Y |
| CH3C.3.2-17 | | L | G | H | V | W | V | G | T | | P | K | S | T | W | Q | Q | G | W |

TABLE 2
Exploration of Acceptable Diversity Within Register and Hot Spot Positions for CH3C.35.21

| | 151 | 152 | 153 | 154 | 155 | 156 | 157 | 158 | 159 | 160 | 161 | 162 | 163 | 164 |
|---|---|---|---|---|---|---|---|---|---|---|---|---|---|---|
| Wild-type | A | V | E | W | E | S | N | G | Q | P | E | N | N | Y |
| CH3C.35.20.1 | . | . | . | . | . | . | F | . | T | E | W | S | S | . |
| CH3C.35.20.2 | . | . | . | . | . | . | Y | . | T | E | W | A | S | . |
| CH3C.35.20.3 | . | . | . | . | . | . | Y | . | T | E | W | V | S | . |
| CH3C.35.20.4 | . | . | . | . | . | . | Y | . | T | E | W | S | S | . |
| CH3C.35.20.5 | . | . | . | . | . | . | F | . | T | E | W | A | S | . |
| CH3C.35.20.6 | . | . | . | . | . | . | F | . | T | E | W | V | S | . |
| CH3C.35.21.a.1 | . | . | W | . | . | . | F | . | T | E | W | S | S | . |
| CH3C.35.21.a.2 | . | . | W | . | . | . | Y | . | T | E | W | A | S | . |
| CH3C.35.21.a.3 | . | . | W | . | . | . | Y | . | T | E | W | V | S | . |
| CH3C.35.21.a.4 | . | . | W | . | . | . | Y | . | T | E | W | S | S | . |
| CH3C.35.21.a.5 | . | . | W | . | . | . | F | . | T | E | W | A | S | . |
| CH3C.35.21.a.6 | . | . | W | . | . | . | F | . | T | E | W | V | . | . |
| CH3C.35.23.1 | . | . | . | . | . | . | F | . | T | E | W | S | . | . |
| CH3C.35.23.2 | . | . | . | . | . | . | Y | . | T | E | W | A | . | . |
| CH3C.35.23.3 | . | . | . | . | . | . | Y | . | T | E | W | V | . | . |
| CH3C.35.23.4 | . | . | . | . | . | . | Y | . | T | E | W | S | . | . |
| CH3C.35.23.5 | . | . | . | . | . | . | F | . | T | E | W | A | . | . |
| CH3C.35.23.6 | . | . | . | . | . | . | F | . | T | E | W | V | . | . |
| CH3C.35.24.1 | . | . | W | . | . | . | F | . | T | E | W | S | . | . |
| CH3C.35.24.2 | . | . | W | . | . | . | Y | . | T | E | W | A | . | . |
| CH3C.35.24.3 | . | . | W | . | . | . | Y | . | T | E | W | V | . | . |
| CH3C.35.24.4 | . | . | W | . | . | . | Y | . | T | E | W | S | . | . |
| CH3C.35.24.5 | . | . | W | . | . | . | F | . | T | E | W | A | . | . |
| CH3C.35.24.6 | . | . | W | . | . | . | F | . | T | E | W | V | . | . |
| CH3C.35.21.17.1 | . | . | L | . | . | . | F | . | T | E | W | S | S | . |
| CH3C.35.21.17.2 | . | . | L | . | . | . | Y | . | T | E | W | A | S | . |
| CH3C.35.21.17.3 | . | . | L | . | . | . | Y | . | T | E | W | V | S | . |
| CH3C.35.21.17.4 | . | . | L | . | . | . | Y | . | T | E | W | S | S | . |
| CH3C.35.21.17.5 | . | . | L | . | . | . | F | . | T | E | W | A | S | . |
| CH3C.35.21.17.6 | . | . | L | . | . | . | F | . | T | E | W | V | S | . |
| CH3C.35.20 | . | . | . | . | . | . | Y | . | T | E | W | S | S | . |
| CH3C.35.21 | . | . | W | . | . | . | Y | . | T | E | W | S | S | . |
| CH3C.35.22 | . | . | W | . | . | . | Y | . | T | E | W | S | . | . |
| CH3C.35.23 | . | . | . | . | . | . | Y | . | T | E | W | S | . | . |
| CH3C.35.24 | . | . | W | . | . | . | Y | . | T | E | W | S | . | . |
| CH3C.35.21.17 | . | . | L | . | . | . | Y | . | T | E | W | S | S | . |
| CH3C.35.N390 | . | . | . | . | . | . | Y | . | T | E | W | S | . | . |
| CH3C.35.20.1.1 | | | | | | | F | | T | E | W | S | S | |
| CH3C.35.23.2.1 | | | | | | | Y | | T | E | W | A | | |
| CH3C.35.23.1.1 | | | | | | | F | | T | E | W | S | | |
| CH3C.35.S413 | | | | | | | Y | | T | E | W | S | S | |
| CH3C.35.23.3.1 | | | | | | | Y | | T | E | W | V | | |
| CH3C.35.N390.1 | | | | | | | Y | | T | E | W | S | | |
| CH3C.35.23.6.1 | | | | | | | F | | T | E | W | V | | |
| | 165 | 184 | 185 | 186 | 187 | 188 | 189 | 190 | 191 | 192 | 193 | 194 | 195 | 196 |
| Wild-type | K | T | V | D | K | S | R | W | Q | Q | G | N | V | F |
| CH3C.35.20.1 | . | . | . | T | . | E | E | . | . | . | . | F | . | . |
| CH3C.35.20.2 | . | . | . | T | . | E | E | . | . | . | . | F | . | . |
| CH3C.35.20.3 | . | . | . | T | . | E | E | . | . | . | . | F | . | . |
| CH3C.35.20.4 | . | . | . | S | . | E | E | . | . | . | . | F | . | . |
| CH3C.35.20.5 | . | . | . | T | . | E | E | . | . | . | . | F | . | . |
| CH3C.35.20.6 | . | . | . | T | . | E | E | . | . | . | . | F | . | . |
| CH3C.35.21.a.1 | . | . | . | T | . | E | E | . | . | . | . | F | . | . |
| CH3C.35.21.a.2 | . | . | . | T | . | E | E | . | . | . | . | F | . | . |
| CH3C.35.21.a.3 | . | . | . | T | . | E | E | . | . | . | . | F | . | . |
| CH3C.35.21.a.4 | . | . | . | S | . | E | E | . | . | . | . | F | . | . |
| CH3C.35.21.a.5 | . | . | . | T | . | E | E | . | . | . | . | F | . | . |
| CH3C.35.21.a.6 | . | . | . | T | . | E | E | . | . | . | . | F | . | . |
| CH3C.35.23.1 | . | . | . | T | . | E | E | . | . | . | . | F | . | . |
| CH3C.35.23.2 | . | . | . | T | . | E | E | . | . | . | . | F | . | . |
| CH3C.35.23.3 | . | . | . | T | . | E | E | . | . | . | . | F | . | . |
| CH3C.35.23.4 | . | . | . | S | . | E | E | . | . | . | . | F | . | . |
| CH3C.35.23.5 | . | . | . | T | . | E | E | . | . | . | . | F | . | . |
| CH3C.35.23.6 | . | . | . | T | . | E | E | . | . | . | . | F | . | . |
| CH3C.35.24.1 | . | . | . | T | . | E | E | . | . | . | . | F | . | . |
| CH3C.35.24.2 | . | . | . | T | . | E | E | . | . | . | . | F | . | . |
| CH3C.35.24.3 | . | . | . | T | . | E | E | . | . | . | . | F | . | . |
| CH3C.35.24.4 | . | . | . | S | . | E | E | . | . | . | . | F | . | . |
| CH3C.35.24.5 | . | . | . | T | . | E | E | . | . | . | . | F | . | . |
| CH3C.35.24.6 | . | . | . | T | . | E | E | . | . | . | . | F | . | . |
| CH3C.35.21.17.1 | . | . | . | T | . | E | E | . | . | . | . | F | . | . |
| CH3C.35.21.17.2 | . | . | . | T | . | E | E | . | . | . | . | F | . | . |
| CH3C.35.21.17.3 | . | . | . | T | . | E | E | . | . | . | . | F | . | . |
| CH3C.35.21.17.4 | . | . | . | S | . | E | E | . | . | . | . | F | . | . |
| CH3C.35.21.17.5 | . | . | . | T | . | E | E | . | . | . | . | F | . | . |
| CH3C.35.21.17.6 | . | . | . | T | . | E | E | . | . | . | . | F | . | . |
| CH3C.35.20 | . | . | . | T | . | E | E | . | . | . | . | F | . | . |
| CH3C.35.21 | . | . | . | T | . | E | E | . | . | . | . | F | . | . |
| CH3C.35.22 | . | . | . | T | . | . | E | . | . | . | . | F | . | . |
| CH3C.35.23 | . | . | . | T | . | E | E | . | . | . | . | F | . | . |
| CH3C.35.24 | . | . | . | T | . | E | E | . | . | . | . | F | . | . |
| CH3C.35.21.17 | . | . | . | T | . | E | E | . | . | . | . | F | . | . |
| CH3C.35.N390 | . | . | . | T | . | . | E | . | . | . | . | F | . | . |
| CH3C.35.20.1.1 | | | | S | | E | E | | | | | F | | |
| CH3C.35.23.2.1 | | | | S | | | E | | | | | F | | |
| CH3C.35.23.1.1 | | | | S | | E | E | | | | | F | | |
| CH3C.35.S413 | | | | S | | | E | | | | | F | | |
| CH3C.35.23.3.1 | | | | S | | E | E | | | | | F | | |
| CH3C.35.N390.1 | | | | S | | | E | | | | | F | | |
| CH3C.35.23.6.1 | | | | S | | E | E | | | | | F | | |

1-25. (canceled)

26. A method for transporting an agent that binds to a therapeutic target across the blood-brain barrier (BBB) of a mammal, comprising exposing the BBB to a protein that binds to a transferrin receptor (TfR) with an affinity of from 400 nM to 2 μM, wherein the protein is linked to the agent and transports the linked agent across the BBB; and wherein the protein is a modified Fc polypeptide that contains a non-native binding site capable of binding TfR.

27. The method of claim 26, wherein the therapeutic target is implicated in a neurodegenerative disease.

28. A method for treating a neurodegenerative disease, comprising administering to a mammal a protein that binds to a transferrin receptor (TfR) with an affinity of from 400 nM to 2 μM, wherein the protein is linked to an agent that binds to a therapeutic target implicated in the neurodegenerative disease; and wherein the protein is a modified Fc polypeptide that contains a non-native binding site capable of binding TfR.

29. The method of claim 26, wherein the protein prolongs brain exposure to the agent as compared to the agent linked to a reference protein that binds to the TfR with a stronger affinity.

30. The method of claim 29, wherein brain exposure is determined by measuring the area under the curve (AUC) of a plot of brain concentration of the agent over time.

31. The method of claim 26, wherein the protein prolongs brain exposure to the agent at a therapeutically effective concentration in the mammal as compared to the agent linked to a reference protein that binds to the TfR with a stronger affinity.

32. The method of claim 26, wherein the TfR is a human TfR.

33. The method of claim 26, wherein the protein binds to the TfR apical domain.

34. The method of claim 26, wherein the protein binds to the TfR with an affinity of from 750 nM to 1.5 μM.

35. The method of claim 26, wherein the protein binds to the TfR with an affinity of from 1,100 nM to 1.5 μM.

36. The method of claim 27, wherein the neurodegenerative disease is selected from the from the group consisting of Alzheimer's disease (AD), Parkinson's disease, amyotrophic lateral sclerosis (ALS), and a combination thereof.

37. The method of claim 28, wherein the protein prolongs brain exposure to the agent as compared to the agent linked to a reference protein that binds to the TfR with a stronger affinity.

38. The method of claim 37, wherein brain exposure is determined by measuring the area under the curve (AUC) of a plot of brain concentration of the agent over time.

39. The method of claim 28, wherein the protein prolongs brain exposure to the agent at a therapeutically effective concentration in the mammal as compared to the agent linked to a reference protein that binds to the TfR with a stronger affinity.

40. The method of claim 28, wherein the TfR is a human TfR.

41. The method of claim 28, wherein the protein binds to the TfR apical domain.

42. The method of claim 28, wherein the protein binds to the TfR with an affinity of from 750 nM to 1.5 μM.

43. The method of claim 28, wherein the protein binds to the TfR with an affinity of from 1,100 nM to 1.5 μM.

44. The method of claim 28, wherein the neurodegenerative disease is selected from the from the group consisting of Alzheimer's disease (AD), Parkinson's disease, amyotrophic lateral sclerosis (ALS), and a combination thereof.


SEQUENCE LISTING (6 sequences)

SEQ ID NO: 1 - protein, 220 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 113 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 3 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 181 aa, Homo sapiens
NSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVS

SEQ ID NO: 5 - protein, 15 aa, Homo sapiens
EPKSCDKTHTCPPCP

SEQ ID NO: 6 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF